(19) United States
(12) Patent Application Publication
(10) Pub. No.: US 2006/0292159 A1
(43) Pub. Date: Dec. 28, 2006

(54) Methods for the inhibition of neovascularization and cancer metastasis

(76) Inventors: Barbara E. Ranscht, Delmar, CA (US); Lionel W. Hebbard, Delmar, CA (US); Robert G. Oshima, Encinitas, CA (US)

Correspondence Address: WOODCOCK WASHBURN LLP, ONE LIBERTY PLACE, 46TH FLOOR, 1650 MARKET STREET, PHILADELPHIA, PA, 19103, US

(21) Appl. No.: 11/450,740

(22) Filed: Jun. 8, 2006

Related U.S. Application Data
(60) Provisional application No. 60/689,133, filed on Jun. 8, 2005.

(51) Int. Cl. G01N 33/574; A61K 39/395

(52) U.S. Cl. 424/155.1; 435/7.23

(57) ABSTRACT

Methods for diagnosing a neoplasia of epithelial tissues are provided. The methods comprise quantifiably detecting T-cadherin expression on tissue samples and comparing the detected T-cadherin levels with reference values for the level of expression of T-cadherin in healthy or disease states. Methods for diagnosing the stage of a neoplasia are also provided. Methods for treating or preventing neovascularization by disrupting the interaction of T-cadherin with adiponectin or other ligands in the vasculature are also provided.

### CROSS-REFERENCE TO RELATED APPLICATIONS

This application claims priority, under 35 U.S.C. §119(e), to Application Ser. No. 60/689,133, filed Jun. 8, 2005, the disclosure of which is incorporated by reference herein in its entirety.

### STATEMENT OF GOVERNMENT SUPPORT

Research leading to the disclosed inventions was funded, in part, with funds from the National Institute of Health, grant number HD25938, the National Cancer Institute, grant numbers CA 098778, CA089140, and U01 CA105490-01, and the Department of Defense, grant numbers W81-XWH-04-1-0574 and DAMD17-02-1-0694. Accordingly, the United States government has certain rights in the inventions described herein.

### FIELD

The invention relates generally to the field of cancer diagnostics and treatments. More specifically, the invention relates to the diagnosis of the presence and stage of progression of various cancers such as breast cancer. The invention also relates more specifically to the treatment of various cancers by inhibiting angiogenesis and neovascularization, and by inhibiting metastasis.

### BACKGROUND

The dependence of tumor growth and progression on the stromal microenvironment has inspired an intense body of research into the identification of factors that regulate these processes (Joyce J A (2005) Cancer Cell 7:513-20). While much of this work has concentrated on vascular factors and inflammatory responses, less is known about the role of adipocyte-derived signals on cancer progression. Adiponectin (also known as ACRP30, AdipoQ, and GBP28) is a fat-secreted, circulating hormone that has been implicated in reducing the risk for obesity-related metabolic disorders such as insulin resistance, hypertension, coronary artery disease, and stroke (Hu E et al. (1996) J. Biol. Chem. 271:10697-703; Maeda K et al. (2002) Biochem. Biophys. Res. Commun. 221:286-9; Scherer P E et al. (1995) J. Biol. Chem. 270:26746-9; Hotta K et al. (2000) Arterioscler. Thromb. Vasc. Biol. 20:1595-9; Iwashima Y et al. (2004) Hypertension 43:1318-23; and, Kumada M et al. (2003) Arterioscler. Thromb. Vasc. Biol. 23:85-9).

Recently, obesity and low levels of adiponectin were correlated with an increased risk of breast cancer in women (Chen D C et al. (2005) Cancer Lett. 11:11; Mantzoros C et al. (2004) J. Clin. Endocrinol. Metab. 89:1102-7). In addition, adiponectin has been associated with endothelial cell functions such as the stimulation of nitric oxide production, the activation of AMP-activated kinase, and Akt signaling (Ouchi N et al. (2000) Circulation 102:1296-1301). Mice with targeted deletions in the adiponectin gene display retarded angiogenic responses, and are affected by metabolic dysregulation (Kubota N et al. (2002) J. Biol. Chem. 277:25863-6; Matsuda M et al. (2002) J. Biol. Chem. 277:37487-91; Shibata R et al. (2005) Nat. Med. 11:1096-1103; Combs T P et al. (2004) Endocrinology 145:367-83; and, Maeda N et al. (2002) Nat. Med. 8:731-7). The cellular and molecular links between the actions of adiponectin and the manifestations of metabolic syndrome, angiogenesis, and cancer have remained elusive.

In search for molecular adiponectin targets, three putative receptors, Adipo 1 and Adipo2 (Yamauchi T et al. (2003) Nature 432:762-9), and T-cadherin (CDH13) (Hug C et al. (2004) Proc. Natl. Acad. Sci. USA 101:10308-13) were identified by expression cloning approaches. T-cadherin was reported as a receptor preferentially binding hexameric and high molecular weight forms of adiponectin (Hug C et al. (2004) Proc. Natl. Acad. Sci. USA 101:10308-13).

T-cadherin is a unique cadherin-type cell adhesion molecule that is anchored to the cell membrane via a glycosylphosphatidylinositol (GPI) moiety (Ranscht B et al. (1991) Neuron 7:391-402; Vestal D J et al. (1992) J. Cell. Biol. 119:451-61). Like other members of the family, T-cadherin is capable of conferring calcium-dependent binding between T-cadherin-expressing cells. In the developing nervous system, T-cadherin demarcates areas of regulated adhesion/de-adhesion in distinct axon pathways and acts as a repulsive cue for population of neuronal growth cones in vitro and in vivo (Fredette B J et al. (1996) Development 122:3163-71; Fredette B J et al. (1994) J. Neurosci. 14:7331-46).

T-cadherin is upregulated in tumor vasculature, and in vitro experiments have implicated T-cadherin in various functions in vascular cells (Oshima R G et al. (2004) Cancer Res. 64:169-79; Wyder L et al. (2000) Cancer Res. 60:4682-8). Similarly, T-cadherin has been reported to interact with adiponectin in vitro (Hug C et al. (2004) Proc. Natl. Acad. Sci. USA 101:10308-13).

Engagement of T-cadherin on cultured endothelial-like cells decreases adhesion and promotes migration through activation of RhoA and Rac (Ivanov D et al. (2004) Cardiovasc. Res. 64:132-43; Ivanov D et al. (2004) Exp. Cell Res. 293:207-18; and, Philippova M et al. (2005) Faseb J. 19:588-90). An upregulation of T-cadherin correlates with neointima formation during experimental restenosis (Kudrjashova E et al. (2002) Histochem. Cell Biol. 118:281-90). In addition, methylation of the T-cadherin promoter is linked to several types of cancers (Fiegl H et al. (2006) Cancer Res. 66:29-33; Hibi K et al. (2004) Br. J. Cancer 91:1139-42; Kim J S et al. (2005) Cancer 104:1825-33; Lewis C M et al. (2005) Clin. Cancer Res. 1 1;166-72; Toyooka K O et al. (2001) Cancer Res. 61:4556-60). The observation that T-cadherin is down-regulated in human mammary cancers has led to the suggestion that T-cadherin can act as a tumor suppressor (Lee S W (1996) Nat. Med. 2:776-82).

Significant complications of tumor growth include vascularization and metastasis. Each of these phenomena represent advanced cancer progression, and in general make eradication of tumors more difficult. As such, one important advance in cancer treatment would be to be able to identify which tumors are about to metastasize. Furthermore, there is a need to inhibit metastasis to facilitate treatment and enhance patient prognosis. Another important advance in cancer treatment would be to inhibit angiogenesis to prevent vascularization of a tumor mass.

### SUMMARY

The invention features methods for diagnosing a neoplasia in a patient that is suspected of having a neoplasia. The methods comprise obtaining a test sample from the patient, such as a tissue biopsy or biological fluid sample, and quantifiably detecting the expression of T-cadherin on the tissue sample, and comparing the detected T-cadherin expression with reference values for T-cadherin expression in subjects with no neoplasia, with a known neoplasia, or both. Comparison with the reference values indicates whether or not the patient has a neoplasia.

The neoplasia can be any neoplasia of any epithelial tissue, and in some embodiments is characterized by diminished T-cadherin expression. Non-limiting examples of neoplasias that are detectable by the inventive methods include breast, esophageal, stomach, lung, cervical, ovarian, bladder, colorectal, gall bladder, pancreatic, or prostate cancer, or hepatocellular carcinoma, cutaneous squamous carcinoma, basal cell carcinoma, lymphoblastic leukemia, malignant B-cell lymphoma, or astrocytoma. The neoplasia can, but need not be metastatic.

Also featured are methods for diagnosing the stage of a neoplasia. The methods comprise obtaining a test sample from the patient, such as a tissue biopsy or biological fluid sample, and quantifiably detecting the expression of T-cadherin on the tissue sample, and comparing the detected T-cadherin expression with reference values for T-cadherin expression in subjects with no neoplasia, with a known stage of neoplasia, or both. Comparison with the reference values indicates the particular stage of the neoplasia in the patient. The stage of the neoplasia can be stage 0, stage I, stage II, stage III, or stage IV as exemplified herein. The methods are applicable to diagnose the stage of any neoplasia, particularly neoplasias of epithelial cells, and more particularly of breast, esophageal, stomach, lung, cervical, ovarian, bladder, colorectal, gall bladder, pancreatic, or prostate cancer, or hepatocellular carcinoma, cutaneous squamous carcinoma, basal cell carcinoma, lymphoblastic leukemia, malignant B-cell lymphoma, or astrocytoma.

The invention also features methods for treating or preventing neovascularization in a patient. The methods can comprise administering to the patient, preferably to the vasculature, a therapeutically effective amount of an inhibitor of T-cadherin expression, wherein the inhibition of T-cadherin expression inhibits the interactions between T-cadherin and adiponectin or interactions between T-cadherin and other ligands in the vasculature, and wherein the inhibition of the interaction between T-cadherin and adiponectin or other T-cadherin ligand prevents neovascularization. The methods can comprise administering to the patient, preferably to the vasculature, a therapeutically effective amount of an inhibitor of the interaction between T-cadherin and its cognate ligand, such as adiponectin. Non-limiting examples of molecules that inhibit the interaction of T-cadherin with its ligands include antibodies to T-cadherin, or antibodies to the ligands, such as antibodies to adiponectin. These methods are applicable to prevent neovascularization of any tissue or organ, or of a neoplasia. The adiponectin can be a fragment of adiponectin, or a monomer or polymer such as a hexamer of adiponectin, or high molecular weight adiponectin. The methods are applicable to treat or prevent neovascularization of an neoplasia as exemplified herein, particularly for breast, esophageal, stomach, lung, cervical, ovarian, bladder, colorectal, gall bladder, pancreatic, or prostate cancer, or hepatocellular carcinoma, cutaneous squamous carcinoma, basal cell carcinoma, lymphoblastic leukemia, malignant B-cell lymphoma, or astocytoma. The methods are also particularly useful to treat or prevent neovascularization of the retina of the eye.

Also featured are methods for identifying modulators of T-cadherin expression. The methods comprise contacting a test compound with a cell expressing T-cadherin and determining an increase or decrease in the expression of T-cadherin on the cell membrane in the presence of the test compound relative to the expression of T-cadherin on the cell membrane in the absence of the test compound. The cell can be any cell that expresses T-cadherin, such as a freshly isolated epithelial cell, or a stable cell or stable cell line that expresses T-cadherin, or a neoplastic cell that expresses T-cadherin. Compounds identified by these methods are deemed to be encompassed within the scope of the present invention.

### BRIEF DESCRIPTION OF THE DRAWINGS

The file of this patent contains at least one drawing/photograph executed in color. Copies of this patent with color drawing(s)/photograph(s) will be provided by the Office upon request and payment of the necessary fee.

FIG. 1 shows T-cadherin localization to the vasculature of the mouse retina. Immunohistochemistry of hypoxic retinae with anti-T-cadherin (A) and CD31 (B) antibodies reveals T-cadherin on the vasculature (C). Scale bar 50 μm. Triple staining for T-cadherin (D), CD31 (E) and NG2 (F) of transversed sectioned retinal vessels and 3 dimensional image reconstruction confirms the expression of T-cadherin on endothelial cells. G shows the complete vessel with endothelial, CD31 and pericyte, NG2 coverage/staining. Colocalization with CD31 (H) but not NG2 (I) defines the endothelial cell localization of T-cadherin (J). Scale bar 5 μm. FIG. 1K shows the vector targeting strategy for the generation of T-cadherin null mice. The 153 bp exon disrupted by insertion of the neomycin cassette corresponds to amino acids 162 to 213 in the extracellular domain 1 of the mouse T-cadherin protein. FIGS. 1 L-U illustrate that T-cadherin^{-/-} mice show restricted angiogenesis after ischemic retinopathy. Biotinylated-BSL1-B4 labeling of normoxic and hypoxic T-cadherin^{+/+} (L and M) and T-cadherin^{-/-} (N and O) retinae reveals reduced staining on hypoxic T-cadherin^{-/-} samples. Scale bar 1 mm. (P) No change in vessel density is observed between T-cadherin^{+/+}=0.01628±0.001 mm2 and T-cadherin^{-/-}=0.017±0.0008 mm2 normoxic retinae. Retinal endothelial cell density is reduced by 48% in T-cadherin-deficient hypoxic retinae (T-cadherin⁺⁺=0.047±0.0015 mm2 to T-cadherin^{-/-}=0.024±0.0015 mm2; P<0.0001). Thirteen sections for each retina from T-cadherin^{-/-} (n=6) and T-cadherin^{+/+} (n=6) mice were analyzed with Image-Pro® software. Visualization of whole mount hypoxic retinal quadrants from T-cadherin^{+/+} (Q) and T-cadherin^{-/-} (R) mice stained with FITC labeled-BSL1-B4, illustrates the changes in retinal angiogenesis. Scale bar 400 μm. Quantitative analysis of hypoxic retinae reveals in T-cadherin-deficient mice: (S) a 63% reduction of proliferative glomeruli; T-cadherin^{+/+}=95.0±7.0, and T-cadherin^{-/-}=35.5±4.5 glomeruli; P<0.0001; T, a 27% reduction of vessel diameter; T-cadherin⁺⁺=6.736±0.2578 μm and T-cadherin^{-/-}=4.937±0.1914 μm, P<0.0001 (three 40× magnified regions were considered for each retina and 15 vessels were measured for each field); and (U) a 53% reduction in vessel branching; T-cadherin^{+/+}=639.3±29.07 and T-cadherin^{-/-}=299.3±11.72 branch points, P<0.0001.

FIG. 2 shows adiponectin binding to the vasculature is contingent on T-cadherin expression. Adiponectin in T-cadherin+/+ hypoxic retinae (A-C) colocalizes with CD31 but is absent from T-cadherin−/− vasculature (D-F). Scale bar 25 μm. Triple staining with adiponectin (G), CD31 (H) and NG2 (I) of hypoxic T-cadherin^{+/+} retinae examined by confocal microscopy and 3-dimensional image reconstruction with the Velocity® program illustrates the expression of adiponectin on endothelial cells. Panel J shows a complete sprouting vessel with CD31-positive endothelial- and NG2-positive pericyte coverage. Comparison of adiponectin with CD31 (K) and NG2 (L) expression confirms the restricted expression of adiponectin on endothelial cells (M). Scale bar 10 μm.

FIG. 3 shows T-cadherin-adiponectin expression in T-cadherin^{+/+} and T-cadherin^{-/-} mammary glands. In 7-week old virgin mouse mammary glands, T-cadherin (A and D) colocalizes with CD31 (B) and SMA a marker for smooth muscle (E), indicating expression on both the endothelium and myoepithelium (C and F). T-cadherin is also observed in the mammary ductal epithelium where it is primarily localized apically. Scale bar 50 μm. Western blot analysis of 11-week old virgin T-cadherin^{+/+} (mouse no. 1223, 1224 and 1225) and T-cadherin^{-/-} (mouse no. 1212, 1213 and 1214) mammary glands shows no apparent differences in the total amount of monomeric adiponectin expression (G) indicating that adiponectin is secreted but cannot be bound to T-cadherin on the vasculature. In the T-cadherin^{+/+} mammary fat pad, adiponectin (H and J) colocalizes with CD31 (I and J), however it is absent from these sites in T-cadherin^{-/-} (K and M) mice (L and M). Scale bar 50 μm.

FIG. 4 shows T-cadherin-deficient MMTV-PyV-mT tumors show restricted growth and reduced endothelial density. A. Time of initial tumor detection by palpation in T-cadherin^{+/+} MMTV-PyV-mT (triangle) and T-cadherin−/− MMTV-PyV-mT (square) transgenic mice is shown as a function of age. The medium time of tumor onset is considered for the two largest tumors and statistically significant by Logrank test (P=0.0268). B and C. Reduction in neoplastic growth in 85-day old whole mount number 4 mammary fat pads in T-cadherin-deficient mice. T-cadherin^{+/+} 245 and T-cadherin^{-/-} 213 mice are supplied as examples. Scale bar, 1 mm. D. Quantification of whole mount mammary glands from 9 animals of each genotype shows a near 3-fold statistically significant reduction in neoplastic area in the knock-out mice (T-cadherin^{+/+} MMTV-PyV-mT (16.74±2.254%) versus T-cadherin^{-/-} MMTV-PyV-mT (5.413±1.627%) of total fat pad area. P=0.0009. E. T-cadherin^{-/-} MMTV-PyV-mT mice extend their life span by 18.5 days as compared to T-cadherin^{+/+} MMTV-PyV-mT transgenic mice. Median survival rates are indicated, and logrank tests indicate a statistically significant difference between the T-cadherin^{+/+} MMTV-PyV-mT and T-cadherin^{-/-} MMTV-PyV-mT lifespan. P=0.0008. F. The mean tumor volume (length×width²)/2 and standard error of the mean is shown as a function of elapsed time after first detection for the largest two tumors of 14 T-cadherin^{+/+} MMTV-PyV-mT and 14 T-cadherin^{-/-} MMTV-PyV-mT mice. Trend lines were determined by linear regression analysis of the means in the Prism statistical program. Linear regression analysis of all individual data points in Prism establishes that the slope difference is significant and that the tumor growth kinetics differ between T-cadherin^{+/+} MMTV-PyV-mT and T-cadherin^{-/-} MMTV-PyV-mT mammary tumors. P<0.0001. G. Immunohistochemistry for phosphorylated histone H3 shows no apparent change in tumor cell proliferation between T-cadherin^{+/+} (H) and T-cadherin^{-/-} (I) MMTV-PyV-mT tumors. Scale bar 50 μm. J, CD31 staining of mammary tumors shows a 31% reduction of tumor endothelial cell area between T-cadherin^{+/+} (K, 5.816±0.4326%) and T-cadherin^{-/-} (L, 4.004±0.3110%) MMTV-PyV-mT tumors. P=0.0022. Scale bar 50 μm. M. TUNEL staining reveals a 6-fold increase in apoptotic tumor nuclei from T-cadherin^{+/+} (N, 1.588±0.4696%) to T-cadherin^{-/-} (O, 10.24±3.105%) MMTV-PyV-mT tumors. P=0.0106. Scale bar 50 μm.

FIG. 5 shows T-cadherin-deficient MMTV-PyV-mT tumor blood vessels lack adiponectin staining. Immunohistochemistry of T-cadherin^{+/+} and T-cadherin^{-/-} MMTV-PyV-mT tumors shows adiponectin on CD31-positive endothelial cells in the wild-type (A-C). However, adiponectin is not recruited to tumor blood vessels in T-cadherin^{-/-} MMTV-PyV-mT tumor blood vessels (D-F).

FIG. 6 shows T-cadherin^{-/-}MMTV-PyV-mT tumors exhibit poorly-differentiated pathology and increased metastatic potential. Examination of hematoxylin and eosin stained sections from mammary tumors and lungs reveals that T-cadherin^{-/-} MMTV-PyV-mT tumors are poorly-differentiated and have greater metastatic potential as than the corresponding wild-type. Examples of gross tumor pathology are shown, A, T-cadherin^{+/+} MMTV-PyV-mT complex solid and papillary carcinoma with prominent vessels and B, three poorly differentiated, partially necrotic T-cadherin^{-/-} MMTV-PyV-mT carcinomas and papillary tumors. Scale bar 1 mm. Magnified images, C and D, of these tumors display glandular forms in differentiated T-cadherin^{+/+}MMTV-PyV-mT tumors and the poorly-differentiated pathology of necrotic T-cadherin^{-/-} tumors. Note the cells undergoing apoptosis either side of the central blood vessel. Scale bar 50 μm. Table 1 supplemental data lists a summary of tumor pathology. Evaluation of the metastatic potential of T-cadherin^{+/+} MMTV-PyV-mT and T-cadherin^{-/-} MMTV-PyV-mT tumors revealed that T-cadherin^{+/+} MMTV-PyV-mT tumors metastasize poorly to the lung (E, 14 mice), whereas all T-cadherin^{-/-} MMTV-PyV-mT tumor-bearing animals (F, 14 mice) show lung metastases. G. Magnified images of the lungs illustrate the invasive phenotype of T-cadherin^{-/-} MMTV-PyV-mT metastasis. Statistics shown in Table 2 supplemental data shows 6.1±4.2 metastases per T-cadherin^{-/-}MMTV-PyV-mT mouse and none in the wild-type. H. Evaluation of the hematoxylin/eosin ratio from mammary tumors shows that T-cadherin^{-/-} MMTV-PyV-mT (35.96±2.556%) tumors exhibit poorly-differentiated pathology as compared to the T-cadherin^{+/+} MMTV-PyV-mT condition (65.35±2.435%) P<0.0001.

FIG. 7 shows a possible model of T-cadherin-adiponectin interaction in angiogenesis. T-cadherin binds adiponectin that in turn can interact with platelet-derived growth factor BB (PDGF-BB, squares), basic fibroblast growth factor (bFGF diamonds) and heparin-binding epidermal growth-like growth factor (HB EGF, circles). Without intending to be bound to any particular theory or mechanism of action, one possible explanation for the angiogenic phenotype of the T-cadherin-deficient mice is that T-cadherin recruits adiponectin to endothelial cells, where it presents growth factors that affect interactions between endothelial cells and pericytes and regulates the assembly of blood vessels.

FIG. 8 shows the ablation of T-cadherin does not effect normal mammary gland ductal outgrowth. Whole mount mammary glands from virgin black 6 T-cadherin^{+/}+ and T-cadherin^{-/-} mice at 4 (A and B), 5 (C and D) and 6 (E and F) weeks of age show no overt defects in mammary gland development. Scale bar 5 mm.

FIG. 9 shows T-cadherin localization to the endothelium in wild-type MMTV-PyV-mT tumors. In T-cadherin^{+/+} PyV-mT tumors T-cadherin (A) is exclusively expressed on the endothelium as evidenced by its colocalization with CD31 (B and C). Scale bar A-C 100 μm.

### DETAILED DESCRIPTION

It is to be understood that this invention is not limited to particular methods, reagents, compounds, compositions or biological systems, which can, of course, vary. It is also to be understood that the terminology used herein is for the purpose of describing particular aspects only, and is not intended to be limiting.

Various terms relating to the methods and other aspects of the present invention are used throughout the specification and claims. Such terms are to be given their ordinary meaning in the art unless otherwise indicated. Other specifically defined terms are to be construed in a manner consistent with the definition provided herein.

As used in this specification and the appended claims, the singular forms “a,” “an,” and “the” include plural referents unless the content clearly dictates otherwise. Thus, for example, reference to “a cell” includes a combination of two or more cells, and the like.

The term “about” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

As used herein, “measure” or “determine” refers to any qualitative or quantitative determinations.

The term “treating” or “treatment” refers to any indicia of success in the attenuation or amelioration of a pathology or condition, including any objective or subjective parameter such as abatement, remission, or reduction of symptoms; increased tolerance by the subject to the pathology or condition; and improved physical or mental well-being of a subject. The indicia of success in the attenuation amelioration of a pathology or condition can be based on any objective or subjective parameters; including the results of a physical examination, neurological examination, and/or psychological or psychiatric evaluations.

“Pharmaceutically acceptable” refers to those properties and/or substances which are acceptable to the patient from a pharmacological/toxicological point of view and to the manufacturing pharmaceutical chemist from a physical/chemical point of view regarding composition, formulation, stability, patient acceptance and bioavailability. “Pharmaceutically acceptable carrier” refers to a medium that does not interfere with the effectiveness of the biological activity of the active ingredient(s) and is not toxic to the host to which it is administered.

As used herein, “test compound” refers to any purified molecule, substantially purified molecule, molecules that are one or more components of a mixture of compounds, or a mixture of a compound with any other material that can be analyzed using the methods of the present invention. Test compounds can be organic or inorganic chemicals, or biomolecules, and all fragments, analogs, homologs, conjugates, and derivatives thereof. Biomolecules include proteins, polypeptides, nucleic acids, lipids, monosaccharides, polysaccharides, and all fragments, analogs, homologs, conjugates, and derivatives thereof. Test compounds can be of natural or synthetic origin, and can be isolated or purified from their naturally occurring sources, or can be synthesized de novo. Test compounds can be defined in terms of structure or composition, or can be undefined. The compound can be an isolated product of unknown structure, a mixture of several known products, or an undefined composition comprising one or more compounds. Examples of undefined compositions include cell and tissue extracts, growth medium in which prokaryotic, eukaryotic, and archaebacterial cells have been cultured, fermentation broths, protein expression libraries, and the like.

“Immunoglobulin” or “antibody” is used broadly to refer to both antibody molecules and a variety of antibody-derived molecules and includes any member of a group of glycoproteins occurring in higher mammals that are major components of the immune system. The term “antibody” is used in the broadest sense and specifically covers monoclonal antibodies, polyclonal antibodies, antibody compositions with polyepitopic specificity, bispecific antibodies, diabodies, and single-chain molecules, as well as antibody fragments (e.g., Fab, F(ab′)2, and Fv), so long as they exhibit the desired biological activity. An immunoglobulin molecule includes antigen binding domains, which each include the light chains and the end-terminal portion of the heavy chain, and the Fc region, which is necessary for a variety of functions, such as complement fixation. There are five classes of immunoglobulins wherein the primary structure of the heavy chain, in the Fc region, determines the immunoglobulin class. Specifically, the alpha, delta, epsilon, gamma, and mu chains correspond to IgA, IgD, IgE, IgG and IgM, respectively. Immunoglobulin and antibody are deemed to include all subclasses of alpha, delta, epsilon, gamma, and mu and also refer to any natural (e.g., IgA and IgM) or synthetic multimers of the four-chain immunoglobulin structure. Antibodies non-covalently, specifically, and reversibly bind an antigen.

A “monoclonal antibody” is an antibody obtained from a population of substantially homogeneous antibodies, i.e., the individual antibodies comprising the population are identical except for possible naturally occurring mutations that can be present in minor amounts. For example, monoclonal antibodies can be produced by a single clone of antibody-producing cells. Unlike polyclonal antibodies, monoclonal antibodies are monospecific (e.g., specific for a single epitope of a single antigen). The modifier “monoclonal” indicates the character of the antibody as being obtained from a substantially homogeneous population of antibodies, and is not to be construed as requiring production of the antibody by any particular method. Screening assays to determine binding specificity of an antibody are well known and routinely practiced in the art. For a comprehensive discussion of such assays, see Harlow et al. (Eds.), ANTIBODIES A LABORATORY MANUAL; Cold Spring Harbor Laboratory; Cold Spring Harbor, N.Y. (1988), Chapter 6.

“Effective amount” refers to an amount of a compound, material, or composition, as described herein effective to achieve a particular biological result. Such results can include, but are not limited to, treating or preventing neoplastic disease, metastasis of a neoplastic disease, and angiogenesis in a subject.

“Tissue” refers to any cell or group of cells.

“Neoplasia” refers to an uncontrolled and/or progressive growth or multiplication of cells or tissues. A “neoplasm” is any group of cells undergoing uncontrolled and/or progressive growth or multiplication. The neoplasia can be benign or malignant. A “neoplastic disease” refers to any condition characterized by a neoplasia.

“Metastasis” refers to the transfer of disease from one organ or part or the body to another organ or part of the body.

“Angiogenic” or “angiogenesis” refers to any formation or development of new or additional blood vessels. “Neovascularization” refers to the formation of new or additional blood vessels in abnormal tissue or in abnormal positions in the body, or in tissues that do not normally contain blood vessels.

“Stable cell” or “stable cell line” refers to any cell in which any portion, including the full-length molecule, of T-cadherin can be expressed.

“High molecular weight adiponectin” refers to polymers of adiponectin that consist of more than two trimers that self-associate to form monomers. This term also encompasses polymers comprising greater than 9 monomers. The basic building block of the adiponectin complex in circulation is the trimeric or low molecular weight adiponectin that is formed by self-assembly of monomeric adiponectin. Adiponectin polymers form from hydrophobic interactions within the globular domain of the molecule. Frequently, two adiponectin trimers self associate to form a disulfide-linked hexamer, generally referred to as the hexameric form of adiponectin. Hexameric adiponectin can further self associate to form higher molecular weight adiponectin. Adiponectin, whether trimeric, hexameric, or high molecular weight, can be post-translationally modified.

It has been discovered accordance with the present invention that T-cadherin expression on vascular endothelial cells is necessary to support tumor angiogenesis and neovascularization. It has been demonstrated for the first time that adiponectin localizes to the vascular endothelium in a T-cadherin manner. Without intending to be bound to any particular theory or mechanism of action, it is believed that his interaction facilitates angiogenesis. This phenomenon is particularly relevant for the neovascularization of tumors, where increased expression of T-cadherin can result in blood vessel formation to support a tumor mass. In addition, it has also been discovered that T cadherin expression diminishes on neoplastic epithelial cells relative to normal cells. This diminished expression is progressive, with further diminution in expression as neoplastic growth advances. As T-cadherin expression decreases, cells become less adherent to each other, thereby facilitating migration and metastasis of neoplastic cells to other locations in the body. Thus, T-cadherin expression in neoplastic transformation occupies two sides of the same coin. On one side, increased T-cadherin expression contributes to malignancy through the promotion of blood vessel formation and vascularization of the growing tumor. On the other side, decreased T-cadherin expression on epithelial cells contributes to malignancy through the promotion of metastasis of the neoplastic cells.

The invention features methods for diagnosing neoplasia in a subject suspected of having a neoplasia. The methods are applicable to detect any neoplasia of any epithelial cell, and particularly applicable to detect any neoplasia having the characteristic of modulated T-cadherin expression. Examples of neoplasias detectable by the inventive methods include, but are not limited to breast, esophageal, stomach, and lung, cervical, ovarian, bladder, colorectal, gallbladder, pancreatic, or prostrate cancers, hepatocellular carcinoma, cutaneous squamous carcinoma, basal cell carcinoma, lymphoblastic leukemia, malignant B-cell lymphoma, astrocytoma, and the like.

In one aspect, the methods comprise obtaining a tissue sample from the subject, quantifiably detecting T-cadherin expression on the tissue sample, and comparing the detected T-cadherin expression with reference values for T-cadherin expression in subjects with no neoplasia, with known neoplasia, or both; the T-cadherin expression relative to the reference values being indicative of the presence or absence of neoplasia, wherein the neoplasia is characterized by diminished T-cadherin expression.

A tissue sample can be obtained from any location in the subject's body where neoplastic cells are suspected to be, or any other location that when screened will provide indicia whether neoplastic cells are present or not in the subject. Tissue samples can be obtained by means of a biopsy or other suitable means in the art. It is also contemplated that T-cadherin expression can be analyzed in biological fluids such as tears, saliva, mucous, whole blood, serum, plasma, urine, bile, secretions, exudate, and the like.

Detection of T-cadherin can be carried out by analyzing cell membranes, or by analyzing cell extracts, or by separating T-cadherin from cell membranes and detecting the separated molecules. Detection of T-cadherin can be carried out using any reagent that specifically recognizes the molecule. Suitable detection reagents will be apparent to those of skill in the art, and can include ligands for T-cadherin such as lipoproteins, including low density lipoprotein (LDL), adiponectin, Eph-receptors such as B1, ephrins such as B1, and the like, non-limiting examples of which are described below. Adiponectin can be monomeric or polymeric (any polymer of at least two adiponectin molecules) including high molecular weight adiponectin (Hug C et al. (2004) Proc. Natl. Acad. Sci. USA. 101:10308-13), or be presented as protein fragments or small molecules that mimic the binding of adiponectin or other ligands to T-cadherin.

Antibodies are a preferred T-cadherin detection reagent. Any antibody that specifically binds to T-cadherin can be used in the present invention. Monoclonal and/or polyclonal antibodies can be used, from whatever source produced, as can recombinant antibodies. Methods for antibody production, including polyclonal, monoclonal, and various recombinant antibodies, are well known to and readily practiced by those of skill in the art. Monoclonal and polyclonal antibodies to T-cadherin are commercially available (see, e.g., catalog number 3583 ProSci Inc, Poway, Calif.; catalog number IMG-5529, Imgenex Corp, San Diego, Calif.), and have been described in the literature and in various patents, see, e.g., U.S. Pat. No. 5,863,804, and 5,585,351, and Takeuchi T et al. (2000) J. Neurochem. 74:1489-97). Monoclonal antibody EC1-4.5, as described in the examples below, is preferred.

Antibodies suitable for use in the methods of the invention include, for example, fully human antibodies, single chain antibodies, human antibody homologs, humanized antibody homologs, chimeric antibodies, chimeric antibody homologs, and monomers or dimers of antibody heavy or light chains or mixtures thereof. The antibodies of the invention can be intact immunoglobulins of any isotype, including types IgA, IgG, IgE, IgD, IgM (as well as all subtypes and idiotypes thereof). The light chains of the immunoglobulin can be kappa or lambda. The antibodies can be portions of intact antibodies that retain antigen-binding specificity, for example, Fab fragments, Fab′ fragments, F(ab′)₂ fragments, F(v) fragments, heavy chain monomers or dimers, light chain monomers or dimers, dimers consisting of one heavy and one light chain, and the like. Recombinant antibodies, including single chain antibodies and phage-displayed antibodies, diabodies, as well as individual antibody light chains, individual antibody heavy chains, chimeric fusions between antibody chains and other molecules, heavy chain monomers or dimers, light chain monomers or dimers, dimers consisting of one heavy and one light chain, and the like, can also be used.

The reagent used to detect T-cadherin can be directly labeled with a detectable moiety. In the alternative, a secondary reagent that specifically recognizes the primary reagent, which is labeled with a detectable moiety is used. The secondary reagent can be any molecule, and is preferably an antibody, including antibodies reactive against LDL and adiponectin. The secondary reagent is labeled with a detectable moiety. Detectable moieties contemplated for use in the invention include, but are not limited to, radioisotopes, fluorescent dyes such as fluorescein, phyocoerythrin, Cy-3, Cy5, allophycocyanin, DAPI, Texas red, rhodamine, Oregon green, lucifer yellow, and the like, green fluorescent protein, red fluorescent protein, Cyan Fluorescent Protein, Yellow Fluorescent Protein, Cerianthus Orange Fluorescent Protein, alkaline phosphatase, β-lactamase, chloramphenicol acetyltransferase, adenosine deaminase, aminoglycoside phosphotransferase (neor, G418r) dihydrofolate reductase, hygromycin-B-phosphotransferase, thymidine kinase, lacZ (encoding β-galactosidase), and xanthine guanine phosphoribosyltransferase, Beta-Glucuronidase, Placental Alkaline Phosphatase, Secreted Embryonic Alkaline Phosphatase, or Firefly or Bacterial Luciferase. Enzyme tags are used with their cognate substrate. As with other standard procedures associated with the practice of the invention, skilled artisans will be aware of additional labels that can be used. In some aspects, the primary reagent or secondary reagent are coupled to biotin, and contacted with avidin or strepatvidin having a detectable moiety tag. Adiponectin is commercially available (see, e.g., BioVision Corp. catalog, Mountain View, Calif.), and methods to label polypeptides are well known in the art. Reagents and methods to label LDL are known in the art, see, e.g., Gordiyenko N et al. (2004) Invest. Ophthalmol. Vis. Sci. 45:2822-9. In addition, labeled LDL is commercially available, see Molecular Probes (Invitrogen) Catalog, Eugene, Oreg.

Reference values for the expression of T-cadherin can be those established for a particular neoplasia such as breast cancer, lung cancer, esophageal cancer, or stomach cancer, or those established for healthy subjects, or both. In addition, the present invention contemplates that screening of patient test samples using the inventive methods will establish new or additional reference values that correlate with disease state or healthy state, including neoplasias newly determined to be characterized by modulated T-cadherin expression, or will provide firmer data with respect to known reference values that correlate with disease and healthy states. Such can serve as additional reference values against which test samples can be compared. Statistically significant modulation of levels of T-cadherin in the test sample relative to the reference values/standards/controls will be indicative of the presence or absence of a neoplastic disease, or will be indicative of the stage of the neoplastic disease.

Cancers are generally clinically classified according to the following scale: Stage 0: The cancer is located only in the inner lining surface of an organ and is not invading the organ. Stage I, where cancer has not spread past the tissue or organ where it started, with relatively small neoplasm. Stage II, where some local and regional spread of cancer, sometimes to lymph nodes, is observed, with increasing size of the neoplasm. Stage III, characterized by extensive local and regional spread of cancer, typically to draining lymph nodes, and still increasing size of the neoplasm. Stage IV, characterized by spread of the cancer (metastasis) beyond the regional lymph nodes to other parts of the body. For the present invention, “stage” of a neoplasia or cancer is intended to refer to this scale.

Normal expression levels of T-cadherin for any given tissue can be empirically determined according to any of various techniques that are known in the art. The normal expression levels can serve as reference values/standards against which the expression levels in patients with suspected neoplastic disease can be compared. Significant deviation (especially negative) over expected normal expression levels of T-cadherin is indicative of the presence or absence, or a particular stage, of neoplastic disease in the patient. In particular, significant diminution of T-cadherin expression indicates that the neoplastic disease is advancing toward metastasis, or is metastatic in the patient. Metastasis can be incipient or progressive. Similarly, the expression levels observed in patients with confirmed neoplastic disease, including confirmed metastatic disease, can also serve as a standard against which the expression levels in patients with suspected disease can be compared. Similar levels of expression of T-cadherin between the known patient and suspected patient is indicative of the presence or absence, or a particular stage, of neoplastic disease in the patient. In such cases, it is expected that the expression level of T-cadherin in both the known and suspected samples will significantly deviate from the level of expression present in healthy subjects.

A variety of assay formats can be used to carry, out the inventive methods, and to quantitatively detect T-cadherin expression. Immunoassays are one assay format, and include but are not limited to ELISA, radioimmunoassays, competition assays, Western blotting, bead agglomeration assays, lateral flow immunoassays, immunochromatographic test strips, dipsticks, migratory format immunoassays, and the like. Other suitable immunoassays will be known to those of relevant skill in the art. In some aspects, microscopy, chromatography including high performance liquid chromatography (HPLC), or spectroscopy can also be used in quantitative detection assays. In some aspects, gel electrophoresis coupled with densitometry is used as the assay.

The general format of the assays involve contacting the reagent with a test sample such as a tissue sample containing the analytes of interest, namely the T-cadherin, which can be distinguished from other components found in the sample. Following interaction of the analyte with the reagent, the system can be washed and then directly detected or detected by means of a secondary reagent as exemplified herein.

In some aspects, the reagent is immobilized on a solid support. In other aspects, the test sample, or molecules separated or purified from the test sample, such as T-cadherin, are immobilized on a solid support. Techniques for purification of biomolecules from samples such as cell membranes, cell lysates, whole cells, tissues, biological fluids, and the like are well known in the art. The technique chosen can vary with the tissue or sample being examined, but it is well within the skill of the art to match the appropriate purification procedure with the test sample source.

Examples of suitable solid supports include, but are not limited to, glass, plastic, metal, latex, rubber, ceramic, polymers such as polypropylene, polyvinylidene difluoride, polyethylene, polystyrene, and polyacrylamide, dextran, cellulose, nitrocellulose, pvdf, nylon, amylase, and the like. A solid support can be flat, concave, or convex, spherical, cylindrical, and the like, and can be particles, beads, membranes, strands, precipitates, gels, sheets, containers, wells, capillaries, films, plates, slides, and the like. The solid support can be magnetic, or a column.

Also featured in the present invention are devices to diagnose a neoplasia in a patient suspected of having a neoplasia. The devices can detect any neoplasia having the characteristic of modulated T-cadherin expression. Examples of neoplasias detectable by the inventive devices include, but are not limited to breast, esophageal, stomach, and lung cancers. The devices can also be used to diagnose a particular stage (0-IV) of a neoplasia. The devices comprise a reagent specific for T-cadherin which reagent is preferably coupled to a solid support. The devices are capable of use in any assay, particularly those described and exemplified herein, wherein the assay can quantifiably detect T-cadherin expression, and wherein modulated levels of T-cadherin expression relative to reference values for T-cadherin expression indicates the presence or absence of a neoplasia, or indicates the particular stage of the neoplasia.

The reagents for use in the devices can be any molecule that specifically binds to T-cadherin, such as those exemplified herein. In some aspects, the reagent is comprised of proteins. In some aspects, the reagent is of antibodies, and any antibody that specifically binds to T-cadherin can be used in the devices. In some aspects, the reagent is a lipoprotein such as LDL. In other aspects, the reagent is adiponectin. Adiponectin can be an adiponectin fragment, a monomer or a polymer, including high molecular weight adiponectin.

The solid support to which the reagent is coupled can be any solid support described herein. The reagent can be immobilized on the solid support by any means suitable in the art, such as adsorption, non-covalent interactions such as hydrophobic interactions, hydrophilic interactions, van der Waals interactions, hydrogen bonding, and ionic interactions, electrostatic interactions, covalent bonds, or by use of a coupling agent. Coupling agents include glutaraldehyde, formaldehyde, hexamethylene diisocyanate, hexamethylene diisothiocyanate, N,N′-polymethylene bisiodoacetamide, N,N′-ethylene bismaleimide, ethylene glycol bissuccinimidyl succinate, bisdiazobenzidine, 1-ethyl-3-(3-dimethylaminopropyl) carbodiimide, succinimidyl 3-(2-pyridyldithio)propionate (SPDP), N-succinimidyl 4-(N-maleimidometyl)cyclohexane-1-carboxylate (SMCC), N-sulfosuccinimidyl 4-(N-maleimidomethyl)-cyclohexane-1-carboxylate, N-succinimidyl (4-iodoacetyl)-aminobenzoate, N-succinimidyl 4-(1-maleimidophenyl)butyrate, N-(epsilon-maleimidocaproyloxy)succinimide (EMCS), iminothiolane, S-acetylmercaptosuccinic anhydride, methyl-3-(4′-dithiopyridyl)propionimidate, methyl-4-mercaptobutyrylimidate, methyl-3-mercaptopropionimidate, N-succinimidyl-S-acetylmercaptoacetate, avidin, streptavidin, biotin, Staphylococcous aureus protein A, and the like.

Sites on the solid support not coupled with the capture reagent can be blocked to prevent non-specific binding of marker molecules to the solid support. Blocking reagents and procedures are well known in the art.

Also featured in accordance with the present invention are kits for diagnosing a neoplasia. The kits can be used to detect any neoplasia having the characteristic of modulated T-cadherin expression. Examples of neoplasias detectable by the inventive kits include, but are not limited to breast, esophageal, stomach, and lung cancers. The kits can also be used to detect a particular stage (0-IV) of the neoplasia. In one aspect, the kits include a reagent that specifically interacts with T-cadherin, and instructions for using the kit in a method for diagnosing a neoplasia having the characteristic of modulated T-cadherin expression, and/or for using the kit in a method for diagnosing the stage of a neoplasia having the characteristic of modulated T-cadherin expression. The reagent can be any molecule that specifically binds to T-cadherin, and the reagent can be directly coupled to a detectable moiety as described herein. In some aspects, where the primary reagent is not coupled to a detectable moiety, the kits can further comprise a secondary reagent that specifically recognizes the primary reagent, wherein secondary reagent is labeled with a detectable moiety. The kits can also include positive and negative controls, and reference values that indicate the presence or absence of the neoplasia, or a particular stage of a neoplasia.

In some aspects, the reagent comprises antibodies, and any antibody that specifically binds to T-cadherin, such as those described herein, can be used in the devices. In some aspects, the reagent is a lipoprotein such as LDL. In other aspects, the reagent is adiponectin. Adiponectin can be a polypeptide fragment of adiponectin, a monomer or polymer, including high molecular weight adiponectin.

In some aspects, the kits further include a solid support to immobilize the reagent or the analyte from the test sample isolated from the patient. The solid support can be any solid support described herein. The kit can further include coupling agents to facilitate immobilization of the reagent or analyte to the solid support. In some aspects, the reagent is provided pre-coupled to the solid support.

The kits can contain materials sufficient for one assay, or can contain sufficient materials for multiple assays.

The invention also features methods for treating or preventing neovascularization in a subject. Diseases characterized by having abnormal or atypical angiogenesis or neovascularization include, but are not limited to, various cancers, including those exemplified herein, diabetic blindness, retinopathy, age-related macular degeneration, rheumatoid arthritis, psoriasis, Kaposi's sarcoma, ischemia, atherosclerosis, and the like. Any disease characterized by neovascularization that is caused, in whole or in part, by the interaction of T-cadherin with adiponectin or other T-cadherin ligands can be treated or prevented using the inventive methods.

In one aspect, the methods comprise administering to a subject having neovascularization a therapeutically effective amount of at least one inhibitor of T-cadherin expression. Administration of the inhibitor of T-cadherin expression prevents or inhibits angiogenesis and neovascularization of the tissue. In some aspects, the inhibitor or inhibitors are administered to blood vessels, particularly to the vascular endothelium, or particular vascular endothelial cells. It is preferable that the inhibitor or inhibitors are administered to or are targeted to cells or tissue in the vasculature that express T-cadherin. In some aspects, the inhibitor or inhibitors of T-cadherin expression are administered in a pharmaceutically acceptable carrier. The inhibitor can target protein expression machinery, for example, the inhibitor can specifically suppress T-cadherin expression from the ribosomes, or can target expressed T-cadherin for proteolysis, such as by ubiquitination of T-cadherin. Alternatively, the inhibitor can target T-cadherin expression at the nucleic acid level.

Expression of the T-cadherin can be specifically suppressed at the nucleic acid level by utilizing antisense nucleic acids or RNA interference (RNAi). A review of RNAi is found in Marx, J. (2000) Science, 288:1370-1372. In brief, traditional methods of gene suppression, employing anti-sense RNA or DNA, operate by binding to the reverse sequence of a gene of interest such that binding interferes with subsequent cellular processes and blocks synthesis of the corresponding protein. Exemplary methods for controlling or modifying gene expression are provided in WO 99/49029, WO 99/53050 and WO 01/75164, the disclosures of which are hereby incorporated by reference in their entirety for all purposes. In these methods, post-transcriptional gene silencing is brought about by a sequence-specific RNA degradation process which results in the rapid degradation of transcripts of sequence-related genes. Studies have shown that double-stranded RNA can act as a mediator of sequence-specific gene silencing (see, for example, Montgomery et al. (1998) Trends in Genetics, 14:255-258). Gene constructs that produce transcripts with self-complementary regions are particularly efficient at gene silencing.

It has been demonstrated that one or more ribonucleases specifically bind to and cleave double-stranded RNA into short fragments. The ribonuclease(s) remains associated with these fragments, which in turn specifically bind to complementary mRNA, i.e., specifically bind to the transcribed mRNA strand for the gene of interest. The mRNA for the gene is also degraded by the ribonuclease(s) into short fragments, thereby obviating translation and expression of the gene. Additionally, an RNA polymerase can act to facilitate the synthesis of numerous copies of the short fragments, which exponentially increases the efficiency of the system. Gene-silencing can extend beyond the cell in which it is initiated such that the inhibition can result in biochemical, molecular, physiological, or phenotypic changes in other cells and systems throughout the organism.

Thus, available genetic information such as the nucleotide sequence, etc. of T-cadherin can be used to generate gene silencing constructs and/or gene-specific self-complementary, double-stranded RNA sequences that can be delivered by conventional art-known methods. A gene construct can be employed to express the self-complementary RNA sequences. Alternatively, cells are contacted with gene-specific double-stranded RNA molecules, such that the RNA molecules are internalized into the cell cytoplasm to exert a gene silencing effect. The double-stranded RNA must have sufficient homology to the targeted gene to mediate RNAi without affecting expression of non-target genes. The double-stranded DNA is at least 20 nucleotides in length, and is preferably 21-23 nucleotides in length. Preferably, the double-stranded RNA corresponds specifically to a polynucleotide of the present invention. The use of small interfering RNA (siRNA) molecules of 21-23 nucleotides in length to suppress gene expression in mammalian cells is described in WO 01/75164. Tools for designing optimal inhibitory siRNAs include that available from DNAengine Inc. (Seattle, Wash.). See WO 01/68836. See also: Bernstein et al., RNA (2001) 7: 1509-21; Bernstein et al., (2001) Nature 409:363-6; Billy et al., (2001) Proc. Nat'l. Acad. Sci. USA 98:14428-33; Caplan et al. (2001) Proc. Nat'l. Acad. Sci. USA 98:9742-7; Carthew et al., (2001) Curr. Opin. Cell Biol. 13: 244-8; Elbashir et al., Nature (2001) 411: 494-8; Hammond et al., Science (2001) 293:1146-50; Hammond et al., (2001) Nat. Ref. Genet. 2:110-9; Hammond et al., (2000) Nature 404:293-6; McCaffrrey et al., (2002) Nature 418-38-39; and McCaffrey et al.,(2002) Mol. Ther. 5:676-84; Paddison et al., (2002) Genes Dev. 16:948-58; Paddison et al., (2002) Proc. Nat'l. Acad. Sci. USA 99:1443-8; Sui et al.,(2002) Proc. Nat'l, Acad. Sci, USA 99:5515-20. U.S. Patents of interest include U.S. Pat. Nos. 5,985,847 and 5,922,687. Also of interest is WO/11092. Additional references of interest include: Acsadi et al., (1991) New Biol. 3:71-81; Chang et al., (2001) J. Virol. 75:3469-73; Hickman et al., (1994) Hum. Gen. Ther. 5:1477-83; Liu et al., (1999) Gene Ther. 6:1258-66; Wolff et al., (1990) Science 247: 1465-8; and Zhang et al., (1999) Hum. Gene Ther. 10:1735-7: and Zhang et al., (1999) Gene Ther. 7:1344-9.

In gene therapy applications, genes are introduced into cells in order to achieve in vivo synthesis of a therapeutically effective genetic product, for example for replacement of a defective gene. “Gene therapy” includes both conventional gene therapy where a lasting effect is achieved by a single treatment, and the administration of gene therapeutic agents, which involves the one time or repeated administration of a therapeutically effective DNA or mRNA. Antisense RNAs and DNAs can be used as therapeutic agents for blocking the expression of certain genes in vivo. It has already been shown that short antisense oligonucleotides can be imported into cells where they act as inhibitors, despite their low intracellular concentrations caused by their restricted uptake by the cell membrane. (Zamecnik et al., (1986) Proc. Natl. Acad. Sci. USA 83:4143-6). The oligonucleotides can be modified to enhance their uptake, e.g., by substituting their negatively charged phosphodiester groups by uncharged groups.

There are a variety of techniques available for introducing nucleic acids into viable cells. The techniques vary depending upon whether the nucleic acid is transferred into cultured cells in vitro, ex vivo, or in vivo in the cells of the intended host. Techniques suitable for the transfer of nucleic acid into cells in vitro include the use of liposomes, electroporation, microinjection, cell fusion, DEAE-dextran, the calcium phosphate precipitation method, etc. In vivo gene transfer techniques include transfection with viral vectors and viral coat protein-liposome mediated transfection (Dzau et al. (1993) Trends in Biotechnology, 11:205-210). Viral vector mediated techniques can employ a variety of viruses in the construction of the construct for delivering the gene of interest. The type of viral vector used is dependent on a number of factors including immunogenicity and tissue tropism. Some non-limiting examples of viral vectors useful in gene therapy include retroviral vectors (see e.g., U.S. Pat. Nos. 6,312,682, 6,235,522, 5,672,510 and 5,952,225,), adenoviral (Ad) vectors (see e.g., U.S. Pat. Nos. 6,482,616, 5,846,945 ) and adeno-associated virus (AAV) vectors (see, e.g., U.S. Pat. Nos. 6,566,119, 6,392,858, 6,468,524 and WO 99/61601 ). In some situations it is desirable to provide the nucleic acid source with an agent that targets the target cells, such as an antibody specific for a cell surface membrane protein or the target cell, a ligand for a receptor on the target cell, and the like. Where liposomes are employed, proteins which bind to a cell surface membrane protein associated with endocytosis can be used for targeting and/or to facilitate uptake, e.g., capsid proteins or fragments thereof tropic for a particular cell type, antibodies for proteins which undergo internalization in cycling, and proteins that target intracellular localization and enhance intracellular half-life. The technique of receptor-mediated endocytosis is described, for example, by Wu et al., (1987) J. Biol. Chem. 262:4429-32; and, Wagner et al., (1990) Proc. Natl. Acad. Sci. USA 87:3410-4. For review of the currently known gene marking and gene therapy protocols see Anderson et al., (1992) Science 256:808-13.

In one aspect, the methods comprise administering to a subject having neovascularization a therapeutically effective amount of at least one inhibitor of T-cadherin biological activity. In one aspect, the methods comprise administering to a subject having neovascularization a therapeutically effective amount of at least one reagent that inhibits the interaction of T-cadherin with adiponectin. The inhibitor that inhibits the interaction of T-cadherin with adiponectin can be specific for or otherwise target T-cadherin, adiponectin, or both. Antibodies to T-cadherin and/or adiponectin are particularly preferred. Antibodies to T-cadherin are described above. Any antibody that specifically binds to adiponectin can be used in the present invention. Monoclonal and/or polyclonal antibodies can be used, from whatever source produced, as can recombinant antibodies. Monoclonal and polyclonal antibodies to adiponectin are commercially available, see, e.g., catalog number MAB3604 and MAB3608 Chemicon, International, Temecula, Calif.; and, Affinity BioReagents catalog, Affinity BioReagents, Golden, Colo. The inhibitors used in these inventive methods will hinder or block the interaction between T-cadherin and adiponectin, or between T-cadherin and any other ligand that promotes T-cadherin-dependent neovascularization, or otherwise block signal transduction such that signals that promote blood vessel formation which are facilitated, or otherwise occur downstream of the interaction of T-cadherin and adiponectin or other T-cadherin ligands in the vasculature are inhibited. Administration of such inhibitors prevents or inhibits angiogenesis and neovascularization of the tissue. In some aspects, the inhibitor or inhibitors are administered to blood vessels, particularly to the vascular endothelium, or particular vascular endothelial cells. It is preferable that the inhibitor or inhibitors are administered to or are targeted to cells or tissue in the vasculature that express T-cadherin. In some aspects, the inhibitor or inhibitors of T-cadherin expression are administered in a pharmaceutically acceptable carrier.

To treat or prevent neovascularization, a therapeutically effective amount of the various inhibitors described above will provide a clinically significant decrease in T-cadherin expression or associated signal pathways in or on vascular endothelial cells, or will inhibit the interaction between T-cadherin and adiponectin such that the signals that promote blood vessel formation are blocked. The inhibitors described above can be administered to any animal, are preferably administered to mammals such as dogs, cats, rats, mice, rabbits, horses, pigs, cows, and donkeys, and are most preferably administered to humans.

The effective amount of the inhibitor(s) of T-cadherin expression, the inhibitor(s) of the interaction of T-cadherin and adiponectin, or the inhibitor(s) of the T-cadherin mediated signals that promote blood vessel formation can be dependent on any number of variables, including without limitation, the species, breed, size, height, weight, age, overall health of the subject, the type of formulation, the mode or manner or administration, or the severity or stage of progression of the pathology such as a neoplasia or retinopathy, or other related condition. The appropriate effective amount can be routinely determined by those of skill in the art using routine optimization techniques and the skilled and informed judgment of the practitioner and other factors evident to those skilled in the art. Preferably, a therapeutically effective dose of the compounds described herein will provide therapeutic benefit without causing substantial toxicity to the subject.

Toxicity and therapeutic efficacy of agents or compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Agents or compositions which exhibit large therapeutic indices are preferred. The dosage of such agents or compositions lies preferably within a range of circulating concentrations that include the ED50 with little or no toxicity. The dosage can vary within this range depending upon the dosage form employed and the route of administration utilized.

For the compositions used in the inventive methods, the therapeutically effective dose can be estimated initially from in vitro assays such as cell culture assays. For example, a dose can be formulated in animal models to achieve a circulating plasma concentration range that includes the IC50 as determined in cell culture (i.e., the concentration of the composition which achieves a half-maximal inhibition of the biological effect in an in vitro assay for angiogenesis. Such information can be used to more accurately determine useful doses in a specified subject such as a human. The treating physician can terminate, interrupt, or adjust administration due to toxicity, or to organ dysfunctions, and can also adjust treatment as necessary if the clinical response were not adequate in order to improve the clinical response.

In some aspects, administration of an inhibitor of T-cadherin expression, an inhibitor of the interaction of T-cadherin and adiponectin, or an inhibitor of the T-cadherin mediated signals that promote blood vessel formation or activity to a subject will achieve a concentration of inhibitor in the range of about 0.1 μM to about 50 mM in the tissues and fluids of the subject, preferably in the vascular endothelium. In some aspects, the range is from 1 μm to 5 mM, in other aspects the range is about 10 μM to 2.5 mM. In still other aspects, the range is about 50 μM to 1 mM. Most preferably the range will be from 1 to 100 μM in the tissues and fluids of the subject, preferably in the vascular endothelium. The concentrations of the components of a pharmaceutical composition which comprises inhibitors of T-cadherin expression or activity are adjusted appropriately to the route of administration, by typical pharmacokinetic and dilution calculations, to achieve such local concentrations.

Treatment can be initiated with smaller dosages that are less than the optimum dose of the T-cadherin inhibitor, followed by an increase in dosage over the course of the treatment until the optimum effect under the circumstances is reached. If needed, the total daily dosage can be divided and administered in portions throughout the day.

For effective inhibition of neovascularization, one skilled in the art can recommend a dosage schedule and dosage amount adequate for the subject being treated. canFor example, dosing can occur one to four times daily for as long as needed. The dosing can occur less frequently if the compositions are formulated in sustained delivery vehicles. The dosage schedule can also vary depending on the active drug concentration, which can depend on the needs of the subject.

The invention also features methods for treating or preventing metastasis of a neoplasia in a subject having a neoplasia. The inventive methods are applicable to treat or prevent metastasis in any neoplasia that is characterized by modulated T-cadherin expression, especially diminished T-cadherin expression, wherein modulated T-cadherin expression facilitates decreased adhesion of neoplastic cells and metastasis of neoplastic cells. In one aspect, the methods comprise administering to the subject a therapeutically effective amount of a compound that inhibits the downregulation of T-cadherin expression on the cell surface. Preferably, the compound inhibits the downregulation of T-cadherin expression on the cell surface of neoplastic cells. In another aspect, the methods comprise administering to the subject a therapeutically effective amount of a compound that facilitates upregulation of T-cadherin expression on the cell surface. Preferably, the compound facilitates the upregulation of T-cadherin expression on the surface of neoplastic cells. Non-limiting examples of neoplasias that can be treated by the inventive methods include breast cancer, esophageal cancer, stomach cancer, lung cancer, and the like.

Administration of such compounds that inhibit downregulation of T-cadherin expression, or of such compounds that facilitate the upregulation of T-cadherin expression is preferably targeted to neoplastic cells, or the situs of a neoplasm in the patient. Factors affecting the amount, duration, route, and the like, of administration are detailed above.

It is preferable that administration of such compounds will achieve a concentration of inhibitor or facilitator in the range of about 0.1 μM to about 50 mM in the tissues and fluids of the subject, preferably in neoplastic cells, and more preferably at the situs of the neoplasm in the subject. In some aspects, the range is from 1 μm to 5 mM, in other aspects the range is about 10 μM to 2.5 mM. In still other aspects, the range is about 50 μM to 1 mM. Most preferably the range will be from 1 to 100 μM in the tissues and fluids of the subject, preferably in the vascular endothelium.

Another aspect of the invention features methods for identifying modulators of T-cadherin expression. In one aspect, the methods comprise contacting a test compound with a cell expressing T-cadherin and determining an increase or decrease in the expression of T-cadherin on the cell relative to the level of expression of T-cadherin in the absence of the test compound. The test compound can be assessed at multiple concentrations, and under varying environmental conditions such as temperature, oxygen, humidity, and the like.

The effects of a test compound on the expression of T-cadherin can be studied on any cell expressing T-cadherin. It is preferable that T-cadherin be expressed on the cell surface. The cells can naturally express T-cadherin, such as cells from the nervous system, heart, skeletal muscle, endothelial cells, smooth muscle cells, and the like. The cells can be neoplastic cells isolated from a patient. The cells can be stable cells or stable cell lines induced to express T-cadherin such as HUVEC cells, (Joshi M B et al. (2005) Faseb J. 19:1737-9), HEK293 cells (Resink T J et al. (1999) FEBS Lett. 463:29-34), Chinese Hamster Ovary cells (Vestal D J et al. (1 992) J. Cell. Biol. 119:451-61), Madin-Darby Canine Kidney (MDCK) cells (Koller E et al. (1996) J. Biol. Chem. 271:30061-7, or L929 cells (Ranscht B unpublished data). Stable cells can be produced by any means suitable in the art for cloning and recombinant gene expression.

The effect of the test compound on the expression of T-cadherin can be determined by any means suitable in the art. The assay used can be qualitative or quantitative. By way of example, and not of limitation, baseline expression of T-cadherin on the untreated cell surface can be measured using labeled antibodies that specifically bind to T-cadherin. In tandem, T-cadherin-expressing cells treated with a test compound can be reacted with the antibodies, and measurements taken to determine whether the levels of T-cadherin expression on the treated cells increased or decreased relative to the baseline. A significant increase or decrease in expression of T-cadherin is indicative that the test compound is effective to modulate T-cadherin expression. Any reagent that specifically binds to T-cadherin can be used, including without limitation, antibodies, lipoproteins, or adiponectin, as exemplified herein. Any assay can be used to detect the reagent bound to T-cadherin, including without limitation, cell-based ELISA, flow cytometry, microscopy (including light microscopy, confocal microscopy, deconvolution microscopy, electron microscopy, and the like), scintillation counting, radiography, and the like. In some aspects, the assay can detect the expression of nucleic acids such as DNA or RNA. For example, and not by way of limitation, modulation of the level of T-cadherin expression can be measured by means of detecting changes in T-cadherin mRNA expression resulting from exposure to the test compound. Suitable nucleic acid-based assays are well known in the art, and include, without limitation, Northern blotting, Southern blotting, gel electrophoresis, polymerase chain reaction, in situ hybridization, microarrays, nucleic acid expression profiles (see, e.g, U.S. Pat. No. 5.958,688), and the like.

The invention also features methods for identifying compounds that modulate T-cadherin expression in a subject by a combination of an in vitro and in vivo screening assay. In one aspect, a test compound is first screened in vitro as described herein, and then screened further in vivo to determine if the compound can modulate T-cadherin expression in the body. After the test compounds are administered to a subject, test samples are periodically taken from the subject and screened to determine if the test compound increased or decreased expression of T-cadherin in tissues within the subject. Once the test sample is taken from the subject, ex vivo screening on the test sample can be practiced according to the details described herein, such as those to practice the diagnostic methods.

Compounds identified by any of the foregoing inventive screening methods are contemplated to be within the scope of this invention. Such compounds can be formulated as a pharmaceutical composition by admixing such compound in an amount effective to modulate the expression of T-cadherin in the subject to which it is administered with a pharmaceutically acceptable carrier, as described herein. Such pharmaceutical compositions can be administered to a subject according to the methods of the invention in order to treat or prevent neovascularization, or to treat or prevent metastasis of a neoplasia.

Pharmaceutically acceptable carriers can be either solid or liquid. Non-limiting examples of solid form preparations include powders, tablets, pills, capsules, lozenges, cachets, suppositories, dispersible granules, and the like. A solid carrier can include one or more substances which can also act as diluents, flavoring agents, buffering agents, binders, preservatives, tablet disintegrating agents, or an encapsulating material. Suitable solid carriers include magnesium carbonate, magnesium stearate, talc, sugar, lactose, pectin, dextrin, starch, gelatin, acacia, tragacanth, methylcellulose, sodium carboxymethyl-cellulose, polyethylene glycols, vegetable oils, agar, a low melting wax, cocoa butter, and the like. Non-limiting examples of suitable disintegrating agents include the cross-linked polyvinylpyrrolidone, agar, or alginic acid or a salt thereof such as sodium alginate. Non-limiting examples of liquid form preparations include solutions, suspensions, syrups, slurries, and emulsions. Suitable liquid carriers include any suitable organic or inorganic solvent, for example, water, alcohol, saline solution, physiological saline, buffered saline, dextrose solution, water propylene glycol solutions, and the like, preferably in sterile form.

The compositions can be formulated and administered to the subject as pharmaceutically acceptable salts. Non-limiting examples of pharmaceutically acceptable salts include acid addition salts such as those containing hydrochloride, sulfate, phosphate, sulfamate, acetate, citrate, lactate, tartrate, methanesulfonate, ethanesulfonate, benzenesulfonate, p-toluenesulfonate, cyclohexylsulfamate and quinate. Such salts can be derived using acids such as hydrochloric acid, sulfuric acid, phosphoric acid, sulfamic acid, acetic acid, citric acid, lactic acid, tartaric acid, malonic acid, methanesulfonic acid, ethanesulfonic acid, benzenesulfonic acid, p-toluenesulfonic acid, cyclohexylsulfamic acid, and quinic acid, according to means known and established in the art.

Also featured are methods to identify compounds that inhibit the interaction of T-cadherin with adiponectin. Also featured are methods to identify compounds that inhibit T-cadherin-mediated signal transduction such that signals that promote blood vessel formation which are facilitated or otherwise occur downstream of the interaction of T-cadherin and adiponectin or T-cadherin and another ligand are inhibited.

Each recited range includes all combinations and sub-combinations of ranges, as well as specific numerals contained therein.

All publications and patent applications cited in this specification are herein incorporated by reference in their entirety for all purposes as if each individual publication or patent application were specifically and individually indicated to be incorporated by reference for all purposes.

Although the foregoing invention has been described in some detail by way of illustration and example for purposes of clarity of understanding, it will be readily apparent to one of ordinary skill in the art in light of the teachings of this invention that certain changes and modifications can be made thereto without departing from the spirit or scope of the appended claims.

The following Exemplary Embodiments of specific embodiments for carrying out the present invention are offered for illustrative purposes only, and are not intended to limit the scope of the present invention in any way.

### EXEMPLARY EMBODIMENTS

### Example 1

### General Experimental Procedures

Animal Models and Tissue Preparation

All experiments were performed in accordance with Burnham Institute of Medical Research Animal Research Committee guidelines in the C57B/6 background.

To isolate a mouse T-cadherin genomic clone, a 129 SVJ mouse genomic library in the lambda FIX@II vector was first screened with a 1020 bp PCR probe which contains the prepeptide to EC3 domain sequence. Twenty two clones were obtained and none of them contained the initiation codon of the mouse T-cadherin. These clones were then screened with a 180 bp PCR probe corresponding to the beginning of the EC1 domain of mouse T cadherin cDNA.

A 14.5 kb long genomic fragment containing an exon of 153 bp corresponding to amino acids 162 to 212 of the mouse T-cadherin protein was isolated and subcloned in the pBluescript IIKS+ vector. The exon was surrounded by two intron sequences: a 5′ arm 4.2 kb long and a 3′ arm 10.2 kb long. 8.5 kb of the sequence located 3′ of the exon has been removed using the unique Cla I site present in the genomic fragment. To insert the neomycine cassette, a Xho I site has been created by point mutation within the exon (Chameleon double-stranded site directed mutagenesis kit). As the new Xho I site needs to be a unique cloning site in the targeting vector, the Xho I site of the pBluescript vector was disrupted at the same time. The neomycine cassette was inserted into the mutated XhoI site within the exon. As a negative selection marker, the blunt-ended herpes simplex virus thymidine kinase gene was inserted close to the end of the 3′ arm of homology, into the blunted Not I site of pBluescript IIKS+. The ScaI linearized targeting vector was electroporated into J1 embryonic stem (ES) cells

Genomic DNA was recovered from ES cell lines and from tail biopsy by incubation overnight at 55 C. in 25 mM EDTA, 50 mM tris pH 8, 0.5% SDS containing 1 mg/ml proteinase K. The RNA was then digested by RNase A 300 mg/ml for 1 hour at 37 C. and after phenol, phenol/chloroform and chloroform extractions, the DNA was ethanol precipitated and resuspended in tris 10 mM, EDTA 1 mM. One tenth of the genomic DNA was digested in the presence of spermidine trihydrochloride 0.01 M, 50 units BamHI enzyme for 5 hours at 37° C. and loaded on a 0.7% agarose gel. The DNA was transferred by capillarity blotting to a Dupont's Polyscreen r PVDF transfer membrane and UV cross linked. The probe was labeled with 32P±dCTP by random priming (Prime-It II Random Primer Labeling kit, Stratagene, La Jolla, Calif.) and the nucleotides removed from the probe by the QIAquick Nucleotide Removal Kit (Qiagen). The probe, 1 kb long, covers a sequence outside the construct. It was obtained by Cla I and BamHI digestion of pBluescript KS+ containing mouse T-cadherin-Sal I-BamHI fragment (5.5 kb). Prehybridization and hybridization were performed in deionized formamide 50%, denhart 5×, 5×SSC, sodium dodecyl sulfate (SDS) 1%, denaturated salmon sperm DNA 100° g/ml. Prehybridization took place at 42° C. for 8 hrs and hybridization (0.5 M cpm/ml) for 16 hrs at 42° C. The blots were washed twice in 0.2×SSC-0.1%SDS at 42° C. for 15 minutes each, followed by a wash at 65° C. for 30 minutes in the same solution. Filters were exposed to Kodak X-OMAT-AR films.

Total RNA was extracted from wild type and knock-out mice tissues using the RNeasy total RNA kit (Qiagen, need full name, city and state). In summary, fresh frozen tissues are first lysed (100 mg tissue/ml lysis buffer) and homogenized 1 minute with a rotor-stator homogenizer under highly denaturated conditions to inactivate RNases and ensure isolation of intact RNA. 4 ml of the sample is adjusted to the appropriate binding conditions and applied to an RNeasy spin column. Total RNA binds, while contaminants are efficiently washed away. High quality RNA is eluted in 300 microliters H₂O.

For Northern blot experiments, 15 μl of total RNA (approximately 10 μg) were resolved on 1% agarose/formaldehyde gels, transferred by capillarity blotting to a nylon membrane (Genescreen, New England Nuclear, Cambridge, Mass.). The RNA were UV cross linked, stained for 15 seconds in 0.02% methylene blue, 0.3 M sodium acetate pH 5.5 and a picture of the membrane was taken to estimate the concentration of RNA loaded in each lane. The probes were labeled with 32P±dCTP by random priming (Prime-It II Random Primer Labeling kit, Stratagen, La Jolla, Calif.) and the nucleotides removed from the probe by the QIAquick Nucleotide Removal Kit (Qiagen). The EC-2 domain through the end of the cDNA probe was obtained in two steps: first, mouse T-cadherin-pBluescript KS+ was digested by Not I-BstE II and the fragment of cDNA remaining religated. This new plasmid was digested by SacI and HindIII to get the nt 842-2200 probe. Prehybridization and hybridization were performed in deionized formamide 50%, 5×SSC, denhart 1×, NaH₂ P0₄ pH 6.5 50 mM, sodium dodecyl sulfate (SDS) 0.2%, salmon sperm DNA 250 jg/ml. Prehybridization took place at 42° C. for 8 hrs and hybridization (2M cpm/ml) for 16 hrs at 42° C. The blots were washed twice in 0.2×SSC-0.1% SDS at 42° C. for 15 minutes each, followed by a wash at 65° C. for 30 minutes in the same solution. Filters were exposed to Kodak X-OMAT-AR films.

For RT-PCR experiments, first strand cDNA was synthesized from wild type and knock-out brain RNA using superscript ‘II RNaseH-, reverse transcriptase (Gibco/BRL) and oligo-dT primers according to the manufacturer's protocol. PCR was carried out with 10% of the first strand reaction, a primer in the exon upstream the targeted exon (nt 432-452) and a primer downstream the targeted exon (nt 784-806).

The linearized T-cadherin gene targeting construct was electroporated into R1 ES cells (from Dr. Nagy, Mt. Sinai Hospital, Toronto, Canada), and clones were selected by resistance to neomycin and insensitivity to ganciclovir. Screening by PCR amplification and Southern analysis identified the targeted allele in 8 clones. Two recombinant ES cell clones were injected into blastocysts of C57B1/6 females and reimplanted into CD1 pseudopregnant females.

Litters from heterozygous matings were genotyped both by PCR and Southern blotting. Southern blotting of BamHI-cut genomic DNA identified the mutated 9 kb DNA fragment in homozygous mutants (−/−), both the 9 kb mutated and the 7.5 kb wildtype DNA fragment in heterozygous animals (±), and the 7.5 kb wildtype DNA fragment in the wildtype (+/+).

Wild-type MMTV-PyV-mT mice were obtained from Dr. William Stallcup at the Burnham Institute. T-cadherin^{-/-} MMTV-PyV-mT mice were derived in two mating steps: i) heterozygous male MMTV-PyV-mT mice were crossed with T-cadherin^{-/-} female mice; and, ii) male MMTV-PyV-mT T-cadherin^{±} progeny was crossed with T-cadherin^{+/}+ and T-cadherin^{-/-} females to yield female MMTV-PyV-mT T-cadherin^{+/+} and PyV-mT T-cadherin^{-/-} mice.

Tumor presence was checked by palpitation twice weekly from 60 days of life and measured with digital calipurs. Tumor volume was calculated according to the following equation (length×width²)/2. Tumor appearance, survival and growth curves were derived in Prisms (GraphPAd, Inc, San Diego, Calif.) using Log-Rank test and Linear Regression analyzes. The animals were housed in the institute vivarium under compliance with the Animal Research Committee.

Antibody Generation, Histochemistry, and Image Analysis

Mice were sacrificed by CO₂ inhalation before removing mammary fat pads or tumors. The tissue was dissected into two halves. One half was fixed in 4% paraformaldehyde in phosphate-buffered saline (PBS) overnight, dehydrated, and embedded in paraffin. Sections were cut at 10 μm and stained with hematoxylin and eosin. The second half was snap-frozen in liquid nitrogen and sectioned frozen at 10 μm for immunohistochemical analysis.

Sections were collected on slides, fixed in ice-cold acetone for 10 minutes and air-dried. Non-specific binding sites were blocked with Tris-buffered saline with 0.05% Tween20 (TBST) buffer containing 10% FCS.

Antibodies to mouse T-cadherin antibodies were generated in rabbits against bacterial T-cadherin-glutathione S-transferase (GST) FP (Amersham Pharmacia Biotech). Mouse T-cadherin cDNA (identical in sequence to GenBank accession number AB022100) was used as a template to amplify DNA encoding EC1 through EC4 and directionally cloned into the bacterial expression vector pGEX-2T. The reading frame of the amplified PCR product was verified by sequencing. The T-cadherin FP was checked for purity and quantity by Coomassie Blue staining on SDS-PAGE gels using bovine serum albumin as a standard.

Two New Zealand white rabbits were immunized and boosted with ˜150 micrograms of T-cadherin GST-FP immobilized on GST-sepharose at 4-wk intervals. For function-blocking experiments, T-cadherin IgG was separated on Protein-A columns and dialyzed against PBS.

Antibodies to CD31 (clone 390, BD PharMingen, San Diego, Calif.), phospho-histone-H3 (Ser10, Upstate Cell Signaling Solutions, Charlottesville, Va.), Acrp30 (adiponectin, PA1-054, Affinity Bioreagents, Golden, Colo.) were purchased from the indicated commercial sources. Anti-NG2 antibodies were a generous gift from Dr. William Stallcup (Burnham Institute, La Jolla).

Sections were incubated with the primary antibodies in TBST overnight. For double immunofluorescence, donkey anti-rabbit streptavidin-Alexa-488 conjugate (Invitrogen Molecular Probes, Carlsbad, Calif.) was used to detect T-cadherin antibodies, anti-rat streptavidin-Alexa-594 conjugate (Molecular Probes) to detect CD31 antibodies and Cy5-conjugated donkey anti-guinea-pig (Jackson ImmunoResearch, West Grove, Pa.) to localize NG2 antibodies. Secondary antibodies were applied for 30 minutes. Apoptotic cells were identified with a commercial terminal deoxynucleotidyl-transferase-mediated nick-labelling immunofluroescence staining kit (ApopAlert™ DNA fragmentation assay kit, BD Biosciences). Sections were counterstained with DAPI (Molecular Probes) for ten minutes and mounted in fluorescent mounting medium (DAKO).

The sections were analyzed by confocal microscopy (MRC-1024 MP Biorad) or constant exposed images were captured with a Spot camera (Diagnostic Incorporated) on a Zeiss Axiovert 405M microscope. Images were analyzed in Photoshop® and three-dimensional images were constructed using Volocity® software. For tumor statistics, four random images of solid tumor from two sections of the largest tumor from each mouse were analyzed. CD31 staining was related to tumor area. Apoptotic and phospho-histone H3 positive cells were related to the total number of DAPI stained nuclei using ImagePro® software. Statistical analyses were performed with Prism® software using the Student's t test on all data sets. Statistics are expressed as mean±standard error of the mean.

Retinal Staining

Retinal neovascularization was induced in T-cadherin^{+/+} and T-cadherin^{-/-} mice using the retinal hypoxia model (Smith et al. (1994) Ivest. Ophthalmol. Vis. Sci. 35:101-11). Mice were euthanized at 17-days-old, and eyes were removed and embedded in OCT (Tissue-Tek®). For quantitation of endothelial cell density, 10 μm serial frozen sections were cut from the middle of the retina to the optic nerve. Frozen sections were treated as above, stained with biotinylated Bandeiraea simplicifolia lectin 1 isolectin B4 (VECTOR Laboratories) for two hours and reacted with ABComplex/HRP (DAKO).

For whole-mount staining, eyes were fixed in 4% paraformaldehyde in PBS overnight and washed in PBS. Retinae were dissected, flat-mounted and permeabilized in PBS containing 10% fetal bovine serum and 0.1% Triton X-100 (PBSFT) overnight. Retinae were stained with FITC-labeled Bandeiraea simplicifolia lectin 1 isolectin B4 (VECTOR Laboratories) in PBSFT overnight, washed five times for one hour each and mounted in Moviol. Image analyses of retinal quadrants were performed with ImagePro software from retinae of 6 mice for each genotype from two separate litters.

Mammary Gland Whole Mounts

Number 4 mammary glands were processed overnight in Carnoy's fixative and stained in Carmine for several hours (http://mammary.nih.gov/tools/histological/Histology/index.html#a1). After dehydration in xylene, glands were mounted with Permount (Fisher Scientific).

Immunoblotting

Tissues were lysed in RIPA buffer (50 mM Tris, pH 7.5, 150 mM NaCl, 1% NP40, 0.5% sodium deoxycholate, 0.1% SDS, 2 mM sodium orthovanadate, 50 mM NaFl, 1 mM sodium molybdate, 40 mM β-glycero-phosphate, 1 mM PMSF and 1/100 protease inhibitor cocktail, SIGMA, P8340), mechanically dissociated by sonication and centrifuged at 14,000 RPM for 10 min at 4° C. The protein concentration of the supernatant was determined by the DC Protein Assay kit (Bio-Rad), reducing sample buffer added and samples were heated to 100° C. for 5 min. A total of 30 μg protein was applied to each lane on SDS-PAGE gels, blotted and incubated with T-cadherin or adiponectin antibodies. Binding of primary antibodies was detected with the ECL Western blotting detection system (Amersham Biosciences).

### Example 2

### T-Cadherin Delineates Vascular Endothelial Cells

To define the vascular localization of T-cadherin, mouse retinae were examined at postnatal day 17 (P17) after hypoxia induced-retinal neovascularization. This model mimics proliferative retinopathy and leads to excessive blood vessel growth and sprouting after sequential exposure of P7 old mice to 75% oxygen followed by normoxic conditions.

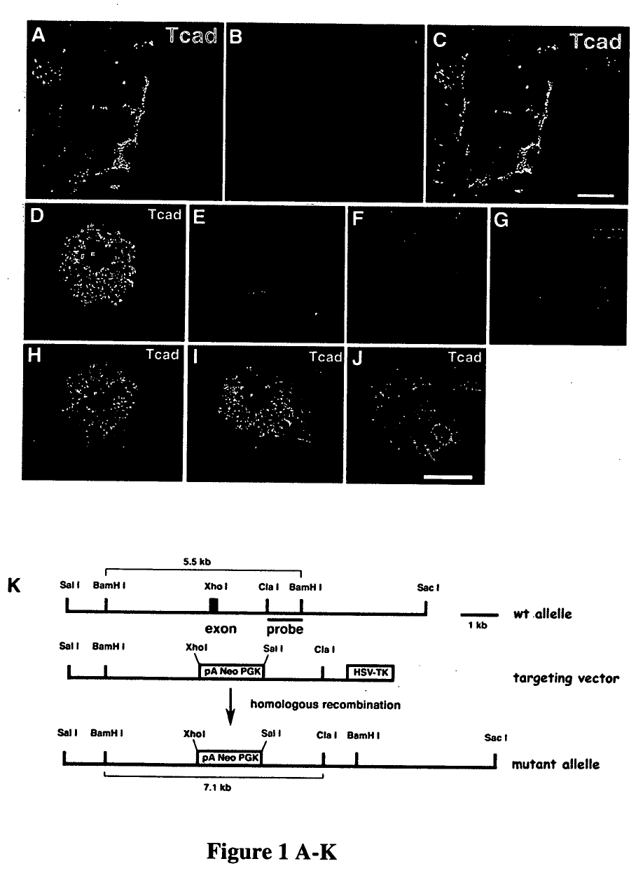

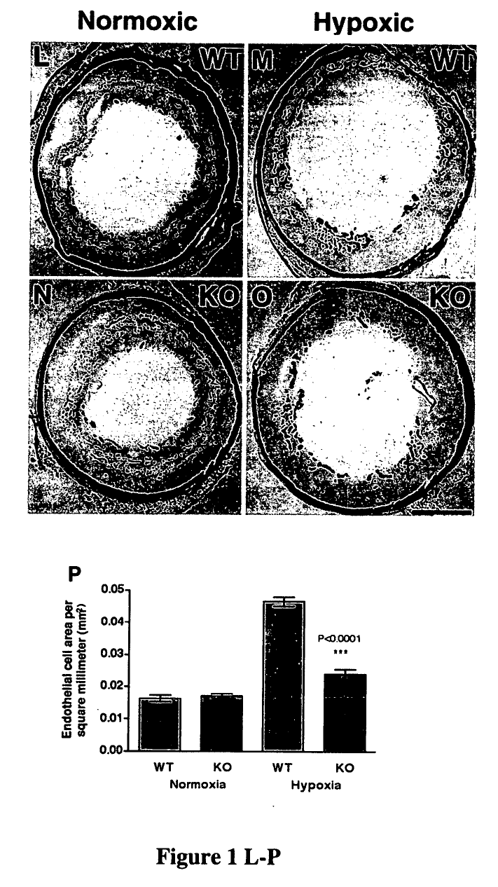

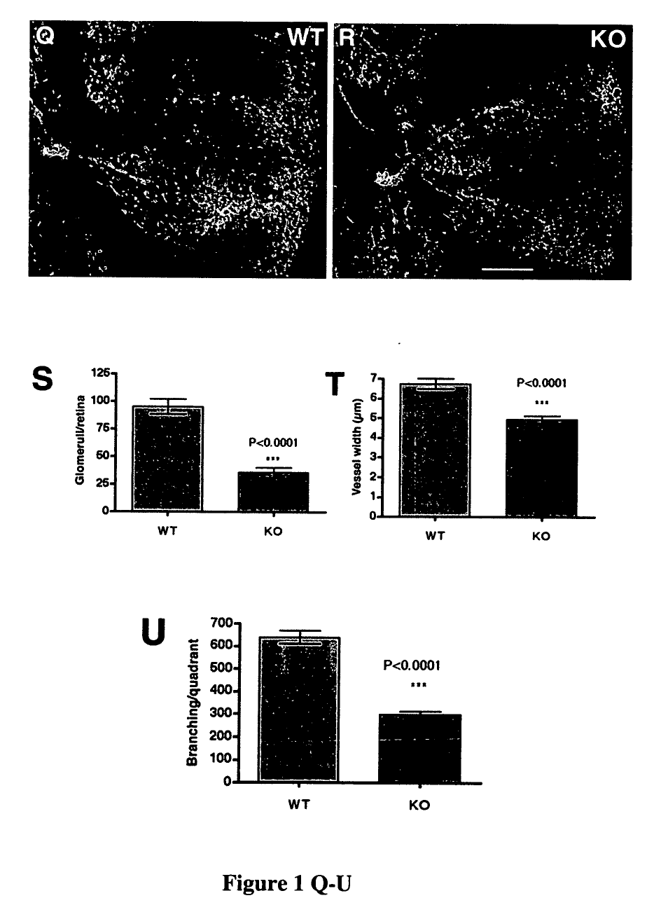

Immunohistochemistry of frozen retina sections revealed prominent T-cadherin expression on the vasculature and less abundant staining in the neuroretina including the plexiform layers and axons of retinal ganglion cells (FIG. 1A). Double immunofluroescence with anti-CD31 and T-cadherin antibodies illustrates the sharp colocalization of T-cadherin with the vascular marker (FIGS. 1B and C). Endothelial cells lining the vasculature are encircled by pericytes that exchange signals with endothelial cells to ensure proper vascularization.

To define the cell types contributing to the T-cadherin-positive vascular staining pattern, T-cadherin immunostaining was combined with the endothelial maker CD31 or the pericyte marker NG2. Labeled sections were examined by confocal microscopy and images of transverse-sectioned retinae were reconstructed three-dimensionally using Velocity® software. Single stained vascular profiles illustrate the distribution of T-cadherin (FIG. 1D), CD31 (FIG. 1E) and the pericyte-specific proteoglycan NG2 (FIG. 1F). The combination of CD31 and NG2 reliably separates endothelial cells and pericytes (FIG. 1G). Overlapping expression with CD31(FIG. 1H) and VE-cadherin (not shown) reveals T-cadherin expression on endothelial cells while complementary expression of T-cadherin and NG2 shows that pericytes are negative for T-cadherin (FIG. 1I and 1J). Taken together, these data establish the restricted expression of T-cadherin on endothelial cells within the vasculature in vivo.

### Example 3

### T-Cadherin-Deficiency Limits Neovascularization in the Retina

To define the role of T-cadherin in angiogenesis, mice deficient for T-cadherin gene expression were generated. The generic insertion targeting vector were engineered to disrupt the T-cadherin extracellular domain 1 after homologous recombination (FIG. 1K). The construct was introduced into embryonic stem cells, and germline chimeras were generated by injection of targeted ES cells into blastocysts.

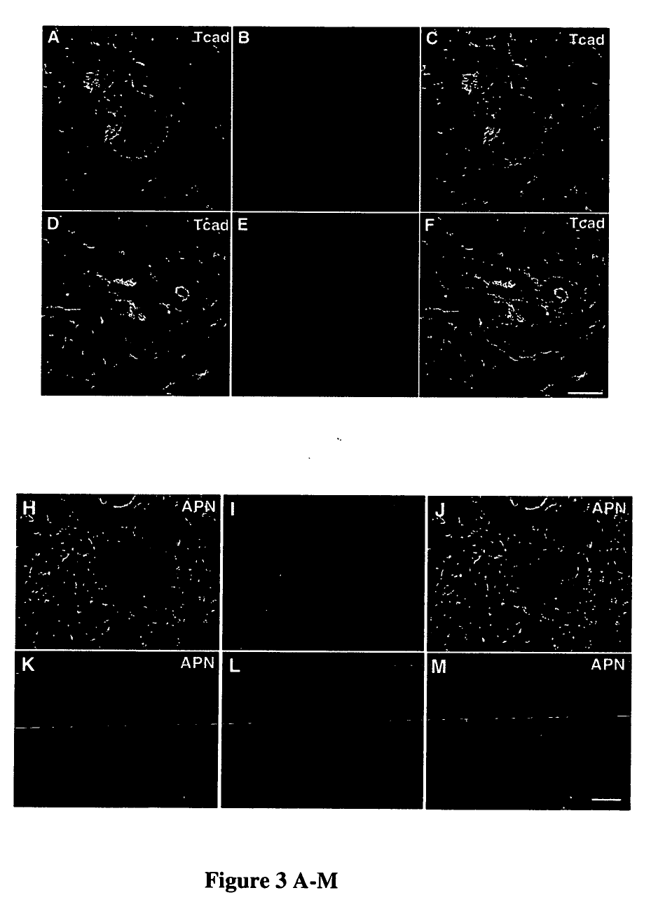

Southern blotting and PCR verified the transfer of the targeted allele (not shown). Mice homozygous for the mutated allele do not express T-cadherin (shown in FIG. 3G for mammary fat pads). The homozygous mutants are viable, have a normal life span and do not spontaneously develop tumors.

Because T-cadherin strongly delineates endothelial cells in the wild-type condition, the vascular pattern in the retina of the T-cadherin^{-/-} mice was investigated. Retinae were examined in sections and flat mounts after labeling, respectively, with biotinylated and FITC labeled Bandeiraea simplicifolia lectin 1 isolectin B4 (BSL1-B4). No overt differences in the retinal vascular pattern were observed between genotypes at P17 (Nornoxic, FIGS. 1L and N).

The mice were then probed for differences in vascular growth after hypoxia exposure. Immunohistochemistry of sequential frozen sections with biotinylated BSL1-B4 showed an apparent reduction in the degree of neovascularization in the T-cadherin mutant condition as compared to the wild type (Hypoxic, FIGS. 1M and O). Quantitative analysis of the area covered by BSL1-B4-staining revealed a decrease of vascular density in the hypoxic retinae from T-cadherin^{-/-} mice by 48% as compared to the wild type (FIG. 1P, P<0.0001).

To further discern the angiogenic differences between genotypes, flat-mounted retinae were stained with FITC labeled-BSL1-B4 and quantified the vascular changes. Images of retinal quadrants showed a clear reduction of vascular density in T-cadherin^{-/-} mice as compared to the T-cadherin^{+/+} condition (FIG. 1Q and R). Closer inspection of the vascular bed revealed that T-cadherin^{-/-} retinae harbor 63% fewer vascular glomeruli, highly proliferative groups of blood vessels protruding through the retinal ganglion cell layer (FIG. 1S). T-cadherin-deficient vessels also showed a 27% decrease in diameter and had 53% fewer branch points in relation to the wild type (FIGS. 1T and U). Without intending to be limited to any particular theory or mechanism of action, these cumulative data suggest the need for T-cadherin in generating adequate vascular responses after hypoxia induction.

### Example 4

### Association of Adiponectin with Vascular Endothelial Cells Depends on T-Cadherin

Binding studies with labeled protein suggest that the adipocye-secreted hormone adiponectin associates with endothelial cells in vivo (Okamoto et al. (2000) Horm. Metab. Res. 32:47-50) and adiponectin binds to T-cadherin in vitro (Hug C et al. 2004), As such, it was investigated whether the localization of adiponectin to vascular cells is dependent on T-cadherin.

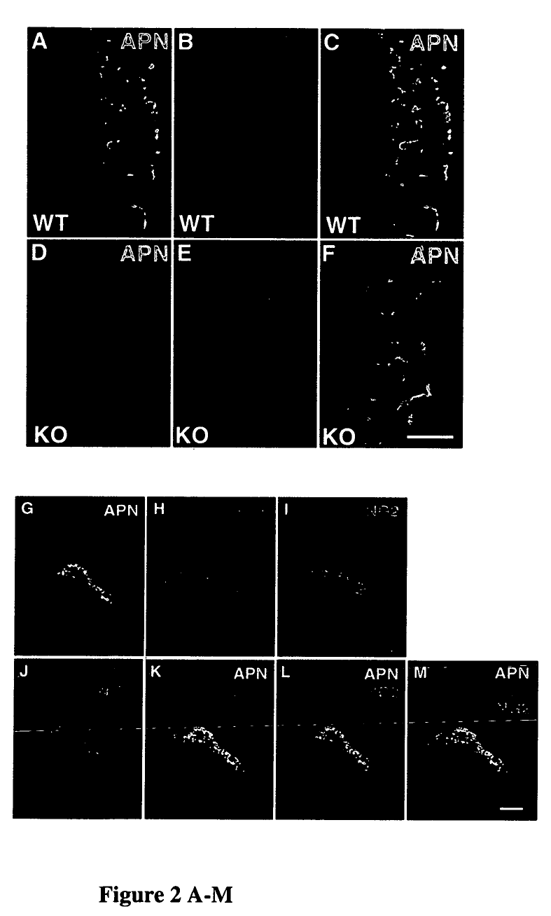

An immunohistochemistry staining protocol was developed for adiponectin on retinal sections. In the wild type condition, adiponectin delineated CD31-positive endothelial cells in P17 hypoxic retinae (FIG. 2A-C).v In contrast, adiponectin was absent from the T-cadherin-deficient retinal vasculature (FIG. 2D-F). vThese data clearly visualize adiponectin on blood vessels in vivo and show that this localisation is contingent on the presence of T-cadherin.

To further clarify the relationship between T-cadherin and adiponectin, the vascular compartment that tethers adiponectin in wild type hypoxic retinae was identified. Utilizing confocal microscopy and three-dimensional image reconstruction in Volocity® it was observed that adiponectin (FIG. 2G) colocalizes with the endothelial cell marker CD31 (FIG. 2H) but not with the pericyte marker NG2 (FIG. 21). FIG. 2J portrays filopodia on the tips of endothelial cells and a pericyte wrapping around the tubular vessel behind the tip cell.

Immunohistochemical staining for adiponectin and CD31 shows a clear colocalization of these proteins (FIG. 2K). In contrast, staining for adiponectin and pericyte-specific NG2 is complementary (FIG. 2L), indicating that in mature vessels adiponectin is associated with endothelial cells and not pericytes (FIG. 2M). These data confirm that T-cadherin and adiponectin are associated within the endothelial compartment of the vasculature where they are positioned to support vascular functions.

### Example 5

### T-Cadherin and Adioponectin Co-Localize on Vascular Endothelial Cells in the Wild-Type Mammary Gland

To identify functions for T-cadherin in the mammary gland, immunohistochemistry was used to determine the localization of T-cadherin in acetone-fixed frozen sections of wild-type virgin mammary glands. T-cadherin was observed on multiple cell types in the mouse mammary fat pad (FIG. 3A and D). Double immunolabeling for T-cadherin and CD31 illustrated the distribution of T-cadherin on vascular endothelial cells (FIG. 3B and C). Staining for T-cadherin and smooth muscle actin (SMA) revealed T-cadherin in the myoepithelium but not in pericytes (FIG. 3E and F). GPI-linked T-cadherin was also detected on the polarized mammary ductal epithelium (FIG. 3A and 3D) where it was distributed weakly at sites of cell-cell contact and strongly demarcated the apical epithelial surfaces pointing towards the ductal lumen.

To confirm the absence of T-cadherin from mammary fat pads in the knockout mice, lysates prepared from mammary fat pads from virgin females were examined using Westen blotting. T-cadherin was detected as the mature 95 kD protein and the 115 kD precursor in the wild-type condition and was absent in the T-cadherin-deficient mice. Reprobing the same blot with antibodies for adiponectin revealed no apparent changes in adiponectin levels between genotypes (FIG. 3G). It should be noted, however, that the reducing conditions for protein separation detect only momomeric adiponectin and are not suited for the identification of specific isoforms.

Probing sections of wild-type and T-cadherin mutant mammary glands with adiponectin antibodies clearly delineated the CD31-positive mammary vasculature in the wild-type mice (FIG. 3H-J). In contrast, no distinct signal was evident for adiponectin in the T-cadherin-deficient condition (FIG. 3K-M). Thus, the expression of adiponectin in the wild-type mammary gland was restricted to vascular cells, and no overlapping signal was detected on the T-cadherin-positive mammary epithelium and myoepithelium. In the T-cadherin-deficient condition, adiponectin was secreted by adipocytes but not recruited to the vasculature.

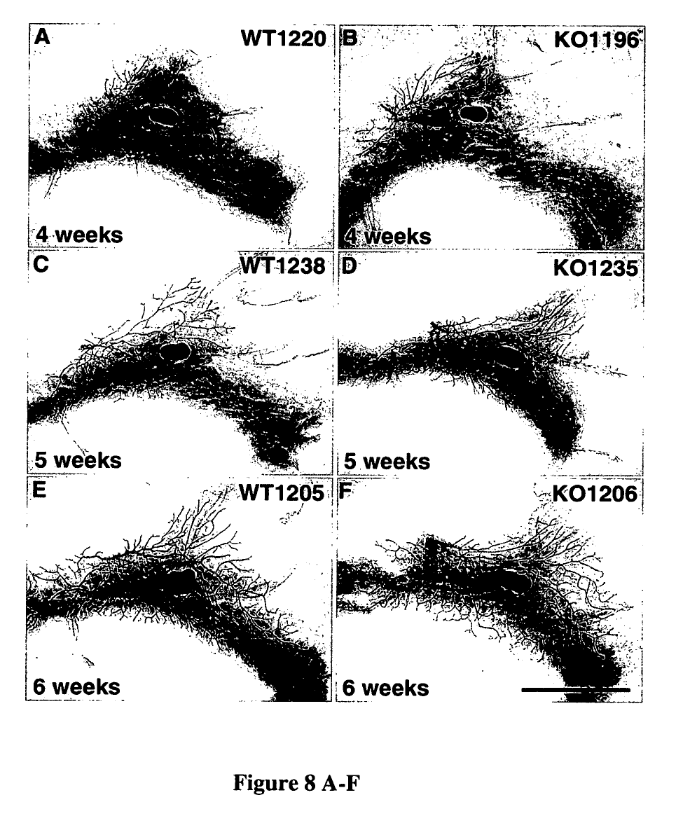

To determine whether the disruption of T-cadherin gene expression is compatible with normal mammary gland development, ductal pattening 4-, 5- and 6-week-old virgin female T-cadherin^{+/+} and T-cadherin^{-/-} mammary glands was analyzed by whole mount carmine staining. Overt differences between genotypes in the degree of mammary ductal growth and branching during puberty were not detected (FIG. 8). Moreover, females deficient for T-cadherin produced and nourished multiple rounds of litters similar in average size to those of wild-type mice. Thus, T-cadherin does not appear to play an essential role in normal mammary gland development.

### Example 6

### T-Cadherin-Deficiency Restricts Tumor Growth in the MMTV-PyV-MT Transgenic Model

Because tumors strongly depend on neovasculariztion for oxygen and nutrient supply, the role of T-cadherin in mammary tumors was investigated. To accomplish this T-cadherin-deficient mice were inter-crossed with transgenic mice expressing the polyma virus middle T antigen from the mouse mammary tumor virus promotor (MMTV-PyV-MT) (Guy CT et al. (1992) Proc. Natl. Acad. Sci. USA 89:10578-82). Depending on the genetic background, the MMTV-PyV-mT model displays in a single primary tumor focus four identifiable stages that closely resemble human breast diseases classified as benign in situ proliferative lesions to invasive carcinomas with a high frequency of distant metastasis (Lifsted T et al. (1998) Int. J. Cancer 77:640-4; Lin E Y et al., (2003) Am. J. Pathol. 163:2113-26).

The expression of bio-markers in the MMTV-PyV-mT-induced tumors, including loss of estrogen and progesterone receptors and integrin-β1, and the increase in leukocyte infiltration accompanied by continued expression of ErbB2/Neu and cyclinD1, closely mimic human mammary tumor characteristics with a poor prognosis (Maglione J E et al. (2001) Cancer Res. 61:8298-8305).

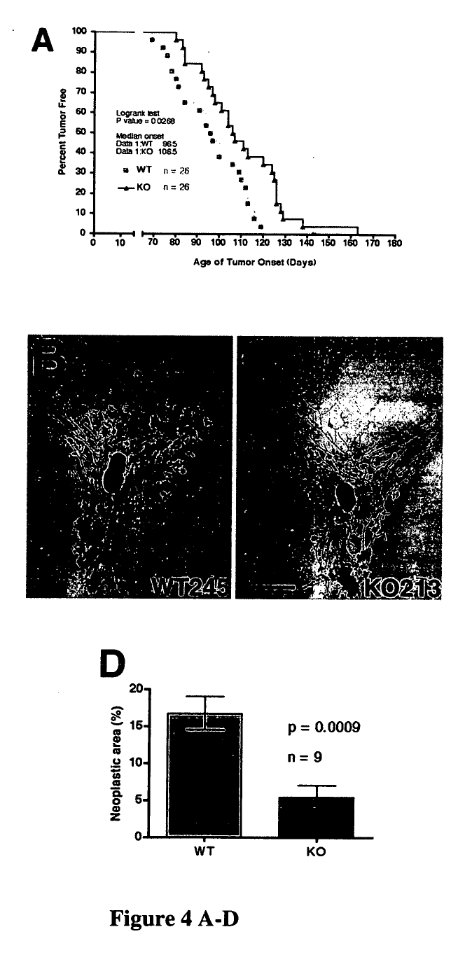

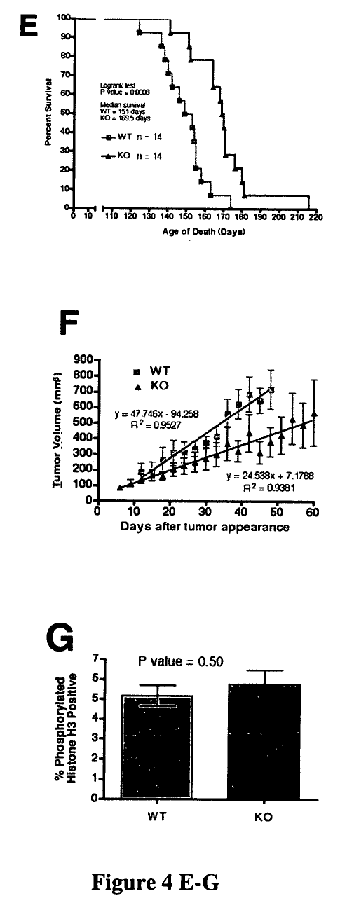

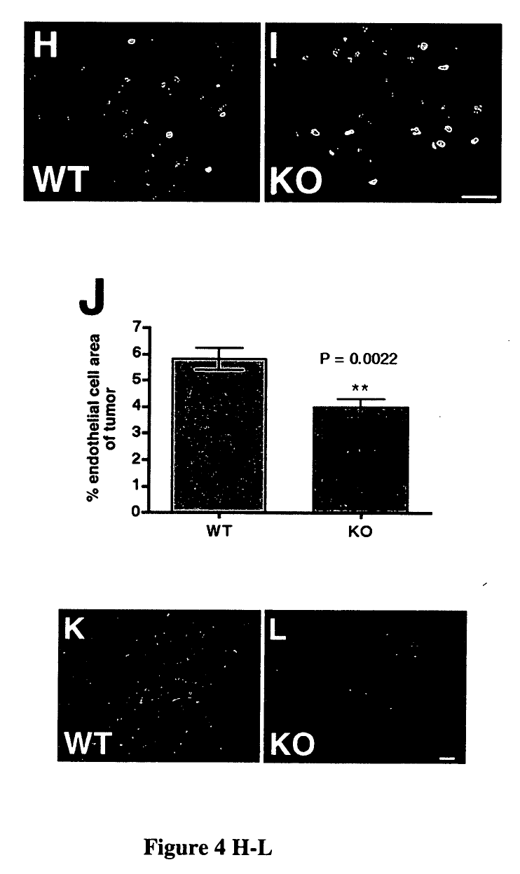

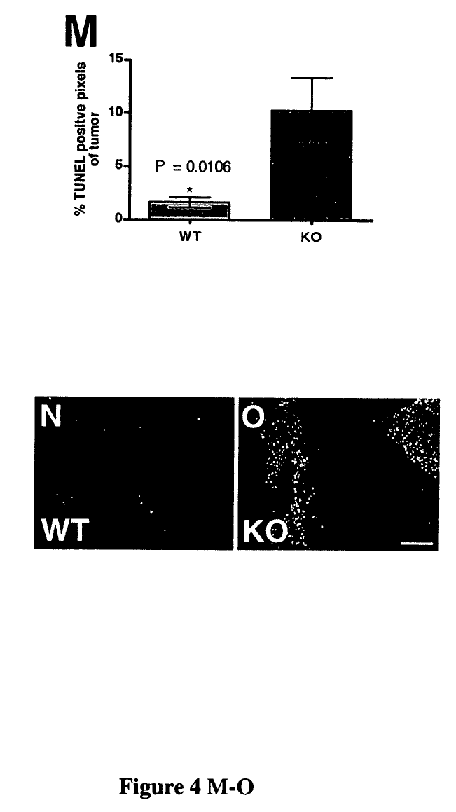

Transgenic MMTV-PyV-MT mice were mated with the T-cadherin null mutants in the syngeneic C57B/6 background and the appearance, size, and progression of mammary tumors in the mice were examined. Palpable tumors were detected in T-cadherin^{-/-} MMTV-PyV-mT mice at a median of 106.5 days compared with a median of 96.5 days for T-cadherin^{+/+} MMTV-PyV-mT mice (FIG. 4A). For closer inspection, the degree of neoplastic growth at 85 days of life in whole mounts of the number four fat pads from both genotypes was examined (FIGS. 4B and C). Images were collected from the two groups of mice and analyzed for neoplastic area using ImagePro®.

T-cadherin^{-/-} MMTV-PyV-mT mice (5.413±1.627%, n=9) displayed a close to three-fold reduction in neoplastic area in the number 4 fat pad as compared to their wild-type counterparts (16.74±2.254%, n=9, FIG. 4D). Additionally, the life span of T-cadherin^{-/-} MMTV-PyV-mT mice was extended from a median of 151 days (T-cadherin^{+/+} MMTV-PyV-mT) to 169.5 days. P=0008 (FIG. 4E). These data suggest that T-cadherin-deficiency delays the appearance of mammary tumors and restricts their growth. In further support for this notion, analyses of the growth kinetics for the two largest tumors in each animal revealed a significant difference in the slope of the tumor growth curves. Regression analysis of all data points collected from the two genotypes confirmed that the difference in slope between the two data sets is very significant with a P-value of P<0.0001 (FIG. 4F). These results provide evidence for the delayed tumor onset and the restriction of tumor growth in T-cadherin^{-/-} MMTV-PyV-mT model as compared to the corresponding wild-type condition.

### Example 7

### Decreased Vascularization and Loss of Adiponectin from the T-Cadherin-Deficient MMTV-PyV-MT Tumor Vasculature

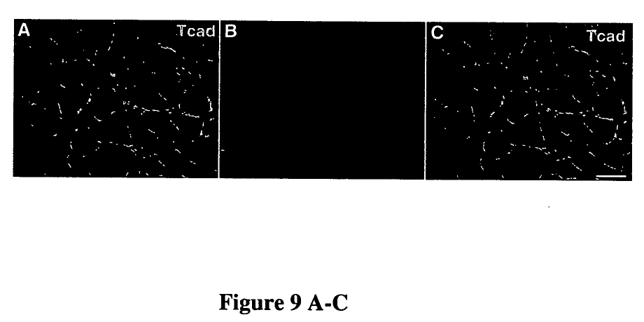

To investigate the cellular events that lead to restricted tumor growth in the T-cadherin-deficient MMTV-PyV-MT model, tumors from both genotypes were analyzed for cell proliferation, blood vessel density and apoptotic rates. First, incorporation of bromodeoxyuridine revealed no changes in the proliferative potential between genotypes. In further support of this observation, no statistical differences in tumor cell proliferation were observed by immunostaining for phosphorylated histone H3 (FIGS. 4G-I). Second, examination of wild-type MMTV-PyV-mT tumors revealed that T-cadherin expression is restricted to the vasculature (FIG. 9). As such, the degree of tumor neovascularization was evaluated by immunostaining with CD31. Endothelial cell density in T-cadherin^{-/-} MMTV-PyV-mT tumors (4.004±0.3110%) was reduced by 31% in comparison with the corresponding wild-type condition (5.816±0.4326%). P=0.0022 (FIGS. 4J-L). Third, because a reduction in tumor vascularization can starve the tumor and kill neoplastic tumor cells, tumor sections from both genotypes were analyzed for DNA strand breaks using Terminal deoxynucleotidyl Transferase Biotin-dUTP Nick End Labeling (TUNEL). A 6-fold increase in apoptotic nuclei was observed in T-cadherin^{-/-} MMTV-PyV-mT tumors (10.24±3.105%) over the corresponding wild-type (1.588±0.4696%). P=0.0106 (FIGS. 4M-O).

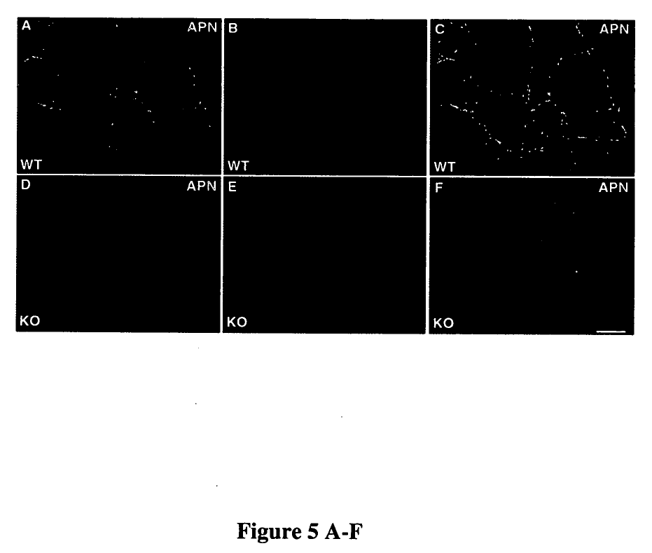

Adiponectin has been implicated in vascular functions and endothelial apoptosis (Kobayashi H et al. (2004) Circ. Res. 94:e27-31; Ouchi N et al. (2004) Circulation 102:1296-1301), and retinal neovascularization is reduced after hypoxia in the T-cadherin^{-/-}mice (FIG. 1). Thus, to link the reduction in blood vessel density with the increase in tumor cell apoptosis in the T-cadherin^{-/-} MMTV-PyV-mT tumors, the distribution of adiponectin was examined. Adiponectin was localized to vascular endothelial cells in T-cadherin^{+/+} MMTV-PyV-mT tumors as determined by colocalisation with CD31 (FIGS. 5A-C), and was absent from these sites in the corresponding T-cadherin^{-/-} mice (FIGS. 5D-F). These results suggest that T-cadherin is necessary for binding adiponectin to the vasculature in MMTV-PyV-mT tumors, and that the disruption of this interaction correlates with the reduction of vascular density and increased cell death.

### Example 8

### Altered Pathology and Metastatic Potential of T-Cadherin-Deficient MMTV-PyV-mT Tumors

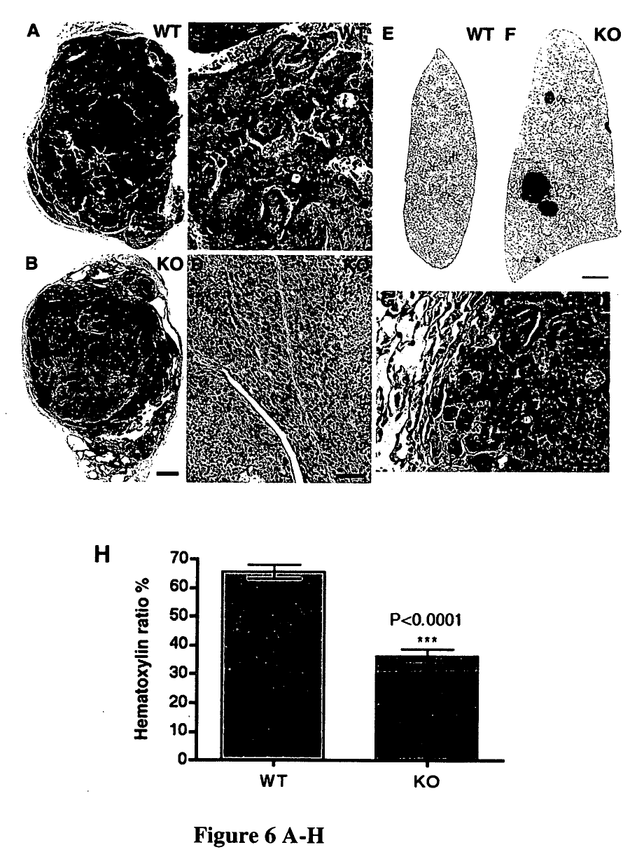

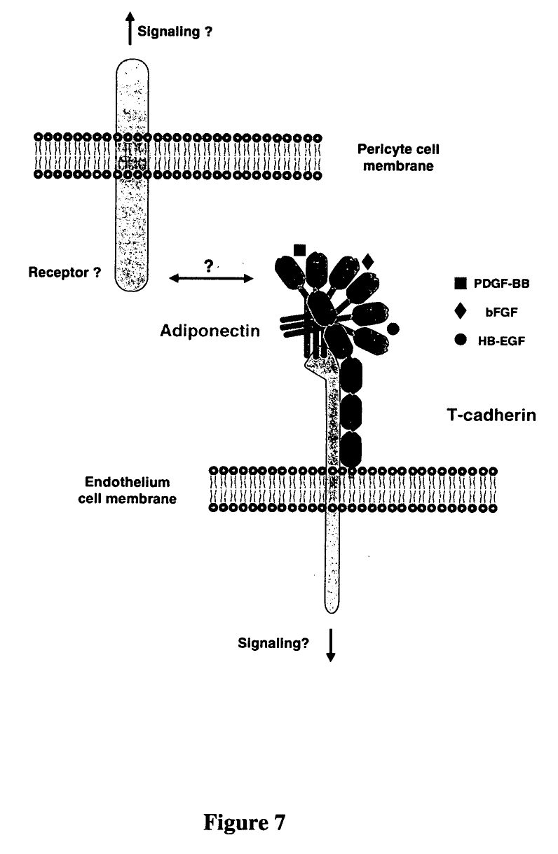

The histology of wild-type and T-cadherin-deficient MMTV-PyV-MT tumors in hematoxilin and eosin stained sections was examined. This analysis revealed an unexpected dramatic change in the tumor characteristics in the T-cadherin^{-/-} mice in terms of aggressiveness and metastatic rate. Eighty-eight tumors from 28 mice were microscopically examined. T-cadherin^{+/+} MMTV-PyV-mT tumors showed the established range of polyoma middle T-induced mammary hyperplasias and mammary tumor phenotypes in the C57B/6 background (Ellies L G et al. (2003) Int. J. Cancer 106:1-7) with a predominance of adenocarcinomas (44/45 tumors) with a mixture of papillary and adenosquamous variants (FIG. 6A and C).

One myoepithelioma was found. The MMTV-PyV-mT-induced tumors in the T-cadherin^{-/-} animals differed from the wild-type cohort with the appearance of a unique poorly-differentiated tumor phenotype (FIGS. 6B and D). This phenotype appeared in 8 of the 14 animals sampled, and in 13 of the 43 (30%) tumor samples (Table 1). The poorly-differentiated tumors are composed of expansile nodular and solid masses of cells with large pleomorphic hyperchromatic nuclei and scanty amphophilic cytoplasm. These cells did not form glands, papillae or squamous areas that are characteristic of the better-differentiated wild-type MMTV-PyV-MT tumors.

TABLE 1
Summary of Pathology Differences.

| Pathology Description | WT Tumors | KO Tumors |
|---|---|---|
| Adenocarcinoma | 25 | 16 |
| Papillary Adenocarcinoma | 11 | 11 |
| Adenosquamous carcinoma | 8 | 2 |
| Poorly differentiated | 0 | 13 |
| adenocarcinoma | | |
| Other | 1 | 1 |

Summary of the cumulative score of pathology evaluations from 45 WT PyV-mT and 43 KO PYV-mt tumors.

The poorly-differentiated tumors in the T-cadherin^{-/-} mice display juxtaposed regions that exhibit a high mitotic rate and areas that are frequently necrotic in line with a decrease in vascularization. All of the T-cadherin-deficient MMTV-PyV-MT tumors developed pulmonary metastases in the C57B/6 background (FIGS. 6E and F). In contrast, none of the wild type MMTV-PyV-MT mice displayed the poorly differentiated tumor phenotype or showed evidence of pulmonary metastases (Table 2). It should be noted that the lung metastatic rate in the MMTV-PyV-mT model depends on the genetic background, and the observation that wild-type MMTV-PyV-MT tumors are much less metastatic in the C57B16 mouse genetic background is in agreement with previous reports (Ellies et al., 2003; Lifsted et al., 1998).

TABLE 2
Lung Metastases.

| Genotype | Mouse No. | Age (days) | Mets/section |
|---|---|---|---|
| WT, PyV-mT | 41 | 138 | 0 |
| WT, PyV-mT | 45 | 158 | 0 |
| WT, PyV-mT | 54 | 142 | 0 |
| WT, PyV-mT | 99 | 149 | 0 |
| WT, PyV-mT | 123 | 155 | 0 |
| WT, PyV-mT | 124 | 158 | 0 |
| WT, PyV-mT | 125 | 155 | 0 |
| WT, PyV-mT | 126 | 140 | 0 |
| WT, PyV-mT | 127 | 174 | 0 |
| WT, PyV-mT | 128 | 146 | 0 |
| WT, PyV-mT | 133 | 153 | 0 |
| WT, PyV-mT | 142 | 124 | 0 |
| WT, PyV-mT | 144 | 163 | 0 |
| WT, PyV-mT | 145 | 136 | 0 |
| KO, PyV-mT | 34 | 164 | 4 |
| KO, PyV-mT | 70 | 470 | 1 |
| KO, PyV-mT | 72 | 164 | 8 |
| KO, PyV-mT | 88 | 141 | 15 |
| KO, PyV-mT | 149 | 171 | 7 |
| KO, PyV-mT | 150 | 171 | 8 |
| KO, PyV-mT | 151 | 151 | 2 |
| KO, PyV-mT | 153 | 216 | 3 |
| KO, PyV-mT | 184 | 152 | 2 |
| KO, PyV-mT | 199 | 180 | 11 |
| KO, PyV-mT | 211 | 176 | 5 |
| KO, PyV-mT | 214 | 168 | 12 |
| KO, PyV-mT | 219 | 181 | 3 |
| KO, PyV-mT | 221 | 169 | 5 |
| KO Average | | | 6.1 +/− 4.2 |

Detection by hematoxylin and eosin staining.
Mets = metastasis;
WT = wild type;
KO = knock out for T-cadherin.

The perimeter of the pulmonary metastases formed by the T-cadherin-deficent tumors was devoid of an endothelial border and metastases were not lodged in the vasculature, suggesting that T-cadherin^{-/-} MMTV-PyV-MT tumors had acquired definitive metastatic properties (FIG. 6G). In further support of the altered tumor phenotype, the hematoxylin:eosin ratio in sections from T-cadherin^{+/+} and T-cadherin^{-/-} MMTV-PyV-MT tumors showed a significant shift in aggressive pathology (increase of pink areas) in T-cadherin^{-/-} PyV-mT tumor-bearing mice (FIG. 6H). P<0.0001. These data suggest that T-cadherin-deficiency induces MMTV-PyV-MT tumors to adopt a malignant phenotype with enhanced metastatic potential.

What is claimed:

1. A method for diagnosing a neoplasia in a subject suspected of having a neoplasia comprising: obtaining a tissue sample from the subject, quantifiably detecting T-cadherin expression on the tissue sample, and comparing the detected T-cadherin expression with reference values for T-cadherin expression in subjects with no neoplasia, with known neoplasia, or both; the T-cadherin expression relative to the reference values being indicative of the presence or absence of neoplasia, wherein the neoplasia is a neoplasia of an epithelial cell.

2. The method of claim 1, wherein the neoplasia is characterized by diminished T-cadherin expression.

3. The method of claim 1, wherein the neoplasia is breast, esophageal, stomach, lung, cervical, ovarian, bladder, colorectal, gall bladder, pancreatic, or prostate cancer, or hepatocellular carcinoma, cutaneous squamous carcinoma, basal cell carcinoma, lymphoblastic leukemia, malignant B-cell lymphoma, or astrocytoma.

4. The method of claim 3, wherein the breast, esophageal, stomach, lung, cervical, ovarian, bladder, colorectal, gall bladder, pancreatic, or prostate cancer, or hepatocellular carcinoma, cutaneous squamous carcinoma, basal cell carcinoma, lymphoblastic leukemia, malignant B-cell lymphoma, or astrocytoma is pre-metastatic.

5. The method of claim 3, wherein the breast, esophageal, stomach, lung, cervical, ovarian, bladder, colorectal, gall bladder, pancreatic, or prostate cancer, or hepatocellular carcinoma, cutaneous squamous carcinoma, basal cell carcinoma, lymphoblastic leukemia, malignant B-cell lymphoma, or astrocytoma is metastatic.

6. The method of claim 1, wherein the tissue sample is breast tissue, esophageal tissue, stomach tissue, lung tissue, cervical tissue, ovarian tissue, bladder tissue, colon tissue, rectal tissue, gall bladder tissue, pancreas tissue, prostate tissue, liver tissue, dermal tissue, epidermal tissue, neural tissue, or lymphocytes.

7. The method of claim 1, further comprising separating T-cadherin from the tissue sample prior to quantifiably detecting the expression of T-cadherin.

8. A method for diagnosing the stage of a neoplasia in a subject comprising obtaining a tissue sample from the subject, quantifiably detecting T-cadherin expression on the tissue sample, and comparing the detected T-cadherin expression with reference values for T-cadherin expression in subjects with no neoplasia, with known neoplasia, or both; the T-cadherin expression relative to the reference values being indicative of the stage of the neoplasia, wherein the neoplasia is a neoplasia of an epithelial cell.

9. The method of claim 8, wherein the neoplasia is characterized by diminished T-cadherin expression.

10. The method of claim 8, wherein the neoplasia is breast, esophageal, stomach, lung, cervical, ovarian, bladder, colorectal, gall bladder, pancreatic, or prostate cancer, or hepatocellular carcinoma, cutaneous squamous carcinoma, basal cell carcinoma, lymphoblastic leukemia, malignant B-cell lymphoma, or astrocytoma.

11. The method of claim 8, wherein the stage is stage 0, stage I, stage II, stage III, or state IV.

12. A method for treating or preventing neovascularization in a subject comprising administering to the vasculature of the subject a therapeutically effective amount of an inhibitor of T-cadherin expression, wherein inhibited T-cadherin expression inhibits the interaction of T-cadherin and adiponectin in the vasculature of the subject and wherein the inhibition of the interaction of T-cadherin and adiponectin prevents neovascularization of a tissue, organ or neoplasm.

13. The method of claim 12, wherein the adiponectin is high molecular weight adiponectin.

14. The method of claim 12, wherein the neoplasm is breast, esophageal, stomach, lung, cervical, ovarian, bladder, colorectal, gall bladder, pancreatic, or prostate cancer, or hepatocellular carcinoma, cutaneous squamous carcinoma, basal cell carcinoma, lymphoblastic leukemia, malignant B-cell lymphoma, or astocytoma.

15. The method of claim 12, wherein the tissue is the retina of the eye.

16. A method for treating or preventing neovascularization in a subject comprising administering to the vasculature of the subject a therapeutically effective amount of a molecule that inhibits the interaction of T-cadherin and adiponectin or T-cadherin with another ligand in the vasculature of the subject, wherein the inhibition of the interaction of T-cadherin and adiponectin or ligand prevents neovascularization of a tissue, organ or neoplasm.

17. The method of claim 16, wherein the molecule is an antibody that specifically binds to T-cadherin.

18. The method of claim 16, wherein the molecule is an antibody that specifically binds to adiponectin.

19. The method of claim 16, wherein the adiponectin is high molecular weight adiponectin.

20. The method of claim 16, wherein the neoplasm is breast, esophageal, stomach, lung, cervical, ovarian, bladder, colorectal, gall bladder, pancreatic, or prostate cancer, or hepatocellular carcinoma, cutaneous squamous carcinoma, basal cell carcinoma, lymphoblastic leukemia, malignant B-cell lymphoma, or astocytoma

21. The method of claim 16, wherein the tissue is the retina of the eye.

22. A method for identifying modulators of T-cadherin expression comprising contacting a test compound with a cell expressing T-cadherin and determining an increase or decrease in the expression of T-cadherin on the cell membrane in the presence of the test compound relative to the expression of T-cadherin in the absence of the test compound.

23. The method of claim 22, wherein the cell is a stable cell or stable cell line expressing T-cadherin.

24. The method of claim 20, wherein the cell is a neoplastic cell.
